(19) United States
(12) Patent Application Publication
(10) Pub. No.: US 2003/0166921 A1
(43) Pub. Date: Sep. 4, 2003

(54) Multielectron redox catalysts

(76) Inventors: Michael J. Therien, Philadelphia, PA (US); James T. Fletcher, San Diego, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/301,017

(22) Filed: Nov. 21, 2002

Related U.S. Application Data
(60) Provisional application No. 60/331,894, filed on Nov. 21, 2001.

(51) Int. Cl. C07D 487/22

(52) U.S. Cl. 540/145

(57) ABSTRACT

This invention relates to synthetic multiporphyrin and multimacrocyclic systems that bind metal ions. Cofacial (porphinato)metal compounds that demonstrate excellent utility in catalyzing multielectron redox transformations are presented. These materials can function as homogeneous or heterogeneous catalysts, or as electrocatalysts when interfaced to an appropriate electrode material.

### CROSS REFERENCE TO RELATED APPLICATIONS

This application claims benefit of provisional application No. 60/331,894 filed Nov. 21, 2001. The entire disclosure of the above-mentioned application is incorporated herein by reference.

### FIELD OF THE INVENTION

This invention relates to synthetic multiporphyrin and multimacrocyclic systems that bind metal ions. A key property of these compositions of matter is their utility in catalyzing multielectron redox transformations. These materials can function as homogeneous or heterogeneous catalysts, or as electrocatalysts when interfaced to an appropriate electrode material.

### BACKGROUND OF THE INVENTION

Though mastered by Nature, the design of biomimetic catalysts satisfying the thermodynamic and kinetic requirements necessary for small molecule multielectron transformations remains challenging. Pioneering studies involving cofacial metallodimacrocycles have provided valuable mechanistic insight regarding a variety of small-molecule reductive and oxidative transformations that require respectively pair wise delivery or removal of electrons. It is noteworthy that cofacial porphyrin constructs, for example, have shown utility as ligands for a wide range of transition-metal-catalyzed multielectron redox transformations of small molecule substrates; well-studied examples include multielectron oxygen and nitrogen reduction reactions, as well as water, hydrogen, and hydrocarbon oxidations. Perhaps the most remarkable aspect of these reactions is that the initial examples of each were all delineated with bimetallic catalysts possessing ligand frameworks having similar electronic structure.

A necessary consequence of utilizing similarly electron rich porphyrin macrocycles in cofacial bis[(porphinato)metal)] catalysts is the fact that these systems often operate at substantial overpotential with respect to the thermodynamic potential of the small molecule multielectron redox conversion of interest. Despite the established catalytic utility of these species, demanding syntheses coupled with limited tools to modulate the electronic properties of these systems have impeded the syntheses of more elaborate, and more thermodynamically efficient versions of the structural motif.

It is therefore desired that new synthetic approaches be brought to bear on this issue. Recently established synthetic routs into 1,2-phenylene bridged cofacial porphyrins that exploit ethyne-bridged multiporphyrin precursors [See, e.g., U.S. Pat. No. 5,798,306, which is incorporated by reference], and sequential Pd-catalyzed cross-coupling and metal-templated [2+2+2] cycloaddition reactions offer a solution to this challenge.

Potentiometric data obtained for materials made by this methodology underscore the utility of this approach and demonstrate that the electronic structure of cofacial bis[(macrocycle)metal] complexes can be modulated to an unprecedented degree. Such potentiometric engineering defines an important tool to exploit in the development of new cofacial porphyrin redox catalysts that operate closer to the substrate thermodynamic potential. Furthermore, it provides the first route into highly electronically asymmetric bis[(macrocyclic)metal] structures that feature predesigned macrocycle-macrocycle lateral shifts; these constructs thus define entirely new opportunities to develop catalysts and electrocatalysts to effect heterolytic bond activation and subsequent multielectron redox transformations of dipolar small molecules.

Numerous porphyrins have been isolated from natural sources. Notable porphyrin-containing natural products include hemoglobin, the chlorophylls, and vitamin B12. Also, many porphyrins have been synthesized in the laboratory, typically through condensation of suitably substituted pyrroles and aldehydes. However, reactions of this type generally proceed in low yield, and cannot be used to produce many types of substituted porphyrins.

### SUMMARY OF THE INVENTION

In one aspect, the instant invention concerns 5,6-Bis(porphinato)zinc(II)indane and derivatives thereof. In some aspects, the porphinato groups are substituted with at least on compound of the group consisting of C₁-C₁₂ alkyl, C₆-C₂₀ aryl, perhaloalkyl, C₂-C₁₂ alkenyl, and C≡C—R; wherein R is H, C₁-C₁₂ alkyl, or C₁-C₂₀ aryl; and said alkyl and aryl groups are optionally substituted with alkyl, aryl, alkoxy, or aryloxy. In this aspect, one embodiment comprises RB substituents at each available methane bridge unit of the porphyrin ring and RA substituents at each available position of the pyrrole ring of the porphyrin ring. Each RA and RB is independently H, C₁-C₁₂ alkyl, C₆-C₂₀ aryl, perhaloalkyl, C₂-C₁₂ alkenyl, and C≡C—R. In some embodiments at least one of the following conditions applies:

(a) at least one of RB₁-RB₈ or one of RA₁-RA₈ is perhaloalkyl; or

(b) at least one of RB₁-RB₈ or one of RA₁-RA₈ is ethyne, alkynyl, oligynyl, or has the formula C≡CR, or

(c) at least one of RA₁-RA₈ is electron withdrawing relative hydrogen provided at least one of RB₁-RB₈ is not H; or

(d) one of RB₁-RB₈ is electron withdrawing relative hydrogen; or

(e) one of RA₁-RA₈ is electron withdrawing relative hydrogen provided all of RA₁-RA₈ are not perfluorophenyl; or

(f) all of RA₁-RA₈ are electron withdrawing relative hydrogen provided at least one of RB₁-RB₈ is not H;

(g) at least one of RB₁-RB₈ or one of RA₁-RA₈ is halooalkyl; or

(h) at least one of RB₁-RB₈ or one of RA₁-RA₈ is ethene, ethynyl, oligoenyl, or has the formula C(R_{C})═C(R_{D})(R_{E}) where R_{C}, R_{D}, and R_{E} are independently H, C₁-C₁₂ alkyl, or C₁-C₂₀ aryl. In another aspect, the invention concerns a compound having formula (1), (2), or (3)

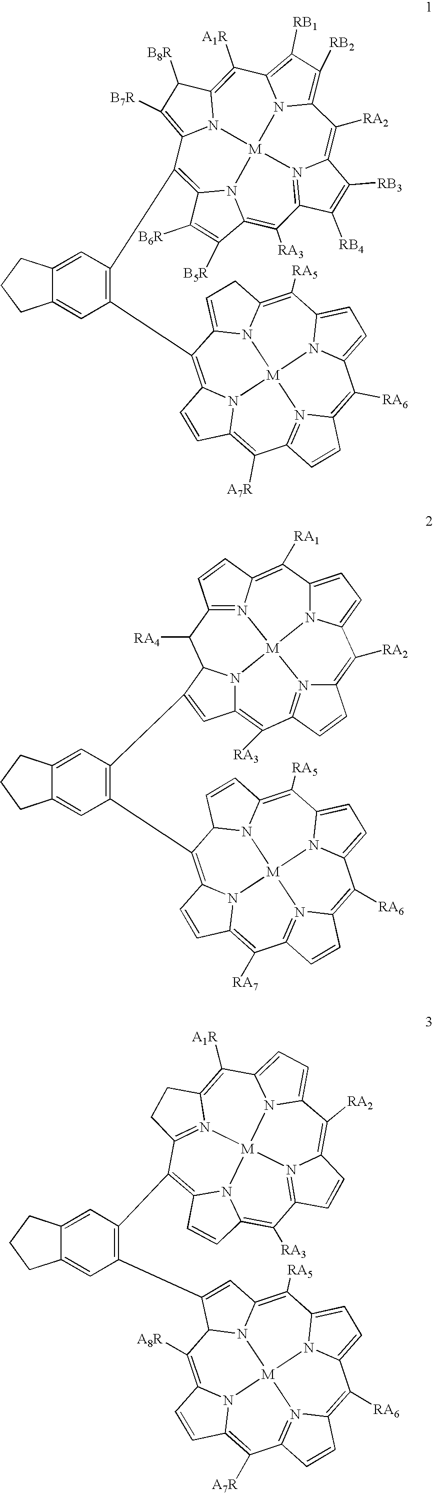

wherein M and M′ are metal ions and RB₁-RB₈ are independently selected from the group consisting of H, C₁-C₁₂ alkyl, C₆-C₂₀ aryl, perhaloalkyl, C₂-C₁₂ alkenyl, and C≡C—R;

wherein R is H, C₁-C₁₂ alkyl, or C₁-C₂₀ aryl;

wherein each alkyl or aryl group may be optionally substituted with alkyl, aryl, alkoxy, or aryloxy;

provided that at least one of the following conditions applies:

(i) at least one of RB₁-RB₈ or one of RA₁-RA₈ is perhaloalkyl; or

(j) at least one of RB₁-RB₈ or one of RA₁-RA₈ is ethyne, alkynyl, oligynyl, or has the formula C≡CR, or

(k) at least one of RA₁-RA₈ is electron withdrawing relative hydrogen provided at least one of RB₁-RB₈ is not H; or

(l) one of RB₁-RB₈ is electron withdrawing relative hydrogen; or

(m) one of RA₁-RA₈ is electron withdrawing relative hydrogen provided all of RA₁-RA₈ are not perfluorophenyl; or

(n) all of RA₁-RA₈ are electron withdrawing relative hydrogen provided at least one of RB₁-RB₈ is not H;

(o) at least one of RB₁-RB₈ or one of RA₁-RA₈ is halooalkyl; or

(p) at least one of RB₁-RB₈ or one of RA₁-RA₈ is ethene, ethynyl, oligoenyl, or has the formula C(R_{C})═C(R_{D})(R_{E}) where R_{C}, R_{D}, and R_{E} are independently H, C₁-C₁₂ alkyl, or C₁-C₂₀ aryl;

In one embodiment, the perhaloalkyl is perfluroalkyl. In some embodiments, M and M′ are zinc.

In other embodiments, the compounds of the instant invention (i) are capable of binding bindstdiatomic, triatomic, or tetraatomic molecules, (ii) have homogeneous or heterogeneous redox catalyst activity, (iii) function as an electrocatalyst, (iv) are capable of selectively oxidizing hydrogen, water, carbon monoxide, or nitric oxide, (v) are capable of selectively oxidizing a hydrocarbon; and/or (vi) are capable of selectively reducing dinitrogen, dioxygen, carbon monoxide, nitric oxide, or carbon dioxide.

In another embodiment, the present invention concerens an electrode wherein one of the compounds of the instant invention is absorbed onto said electrode. In further embodiments, the compound may be covalently or noncovalently bonded to the electrode.

In another embodiment, the instant invention concerns a fuel cell comprising a compound described herein.

In one embodiment, the invention concerns a method comprising an oxidative or reductive transformation comprising contacting a reactant with a compound of the instant invention. In another aspect, the invention concerns a method for effecting an oxidative or reductive transformation comprising contacting a reactant with a compound of the instant invention.

### BRIEF DESCRIPTION OF THE DRAWINGS

The numerous objects and advantage of the present invention can be better understood by those skilled in the art by reference to the accompanying figures, in which:

FIG. 1 shows the synthesis of the extremely electron-poor ethyne-linked dimeric (porphinato)zinc(II) species, bis[([(2,2′-5,10,15,20-trifluoromethyl]porphinato)zinc(II)]ethyne (18).

FIG. 2 shows the conversion of ethyne-bridged, bis[(porphinato)zinc(II)] species 12-18 to their corresponding 1,2-phenylene-bridged bis[(porphinato)metal compounds.

FIG. 3 illustrates possible meso- enantiomers and - atropisomers in cofacial bis[(porphinato)zinc(II)] complexes featuring these linkage topologies.

FIG. 4 shows the synthesis of 5-(6-phenyl)indanyl-derivatized porphyrin monomers.

FIG. 5 shows electronic absorption spectra of cofacial bis[(porphinato)zinc(II)] complexes.

FIG. 6 shows potentiometrically determined relative frontier molecular orbital energy levels.

FIG. 7 shows potentiometrically determined relative frontier molecular orbital energy levels.

FIG. 8 is a qualitative depiction of (porphinato)zinc(II)-(porphinato)zinc(II) HOMO-HOMO interactions occurring within van der Waals contact in cofacial porphyrin systems possessing idealized 60 degree dihedral angles between porphyrin least-squares planes.

FIG. 9 is a is a qualitative depiction of (porphinato)zinc(II)-(porphinato)zinc(II) LUMO-LUMO interactions occurring within van der Waals contact in cofacial porphyrin systems possessing idealized 60 degree dihedral angles between porphyrin least-squares planes.

FIG. 10 shows examples of Co-mediated [2+2+2] cycloaddition reactions of ethyne-containing porphyrin structures with 1,6-heptadiyne.

FIG. 11 shows cofacial bis[(porphinato)metal] catalysts for the reduction of dioxygen.

FIG. 12 shows electric modulation via perfluoroalkyl substitution.

FIG. 13 is a schematic of redox energies of porphyrin monomers and dimers.

FIG. 14 demonstrates the changes observed in cyclic voltammetric responses upon dioxygen binding.

### DETAILED DESCRIPTION OF ILLUSTRATIVE EMBODIMENTS

(Porphinato)metal species bearing metal complexes or other redox active entities as macrocycle substituents have been examined for their potential utility as multielectron oxidation and reduction catalysts. In these systems, the porphyrin-pendant substituents are envisaged to serve as electron reservoirs or sinks, delivering or removing multiple electrons from the focal catalytic unit during substrate turnover. While the utility of such assemblies has been demonstrated, in practice, less than optimal electronic coupling between the redox active moieties and the catalytic core often limits the catalytic effectiveness of these supramolecular species.

With respect to small molecule activation, the cofacial (porphinato)metal structural motif has shown particular catalytic efficacy in a wide range of multielectron redox transformations. This catalytic diversity derives from both the rich coordination chemistry of redox-active metalloporphyrin compounds, and the ease at which appropriate metal-metal distances can be accommodated in such structures. Compounds disclosed herein new archetypes that feature substantial conjugation expansion of the classic cofacial bis(porphyrin) structural motif. These multiporphyrin compounds exploit a face-to-face bis(porphinato)metal core and auxiliary (porphinato)metal units that are linked to this structure via an ethyne-based macrocycle-to-macrocycle linkage topology, a mode of porphyrin-to-porphyrin connectivity that enables exceptional ground- and excited-state electronic communication between porphyrin centers. Such oligo[(porphinato)metal] structures, featuring extensive porphyrin-porphyrin frontier orbital interactions enabled by both π-stacking and the cylindrically π-symmetric ethyne moiety, define potentially attractive ligand platforms from which to evolve new multielectron redox systems.

The fabrication of these species may take advantage of a new synthesis of 1,2-phenylene-bridged cofacial porphyrins that exploits ethyne-bridged multiporphyrin precursors constructed via sequential Pd-catalyzed cross-coupling and metal-templated [2+2+2] cycloaddition reactions. In this patent, we utilize this chemistry to synthesize face-to-face bis[(porphinato)metal] species that bear macrocycle-ethyne substituents; further application of Pd-catalyzed cross-coupling methodology extends conjugation of the cofacial (porphinato)metal core to additional porphyrin units, giving novel tris- and tetrakis[(porphinato)metal] compounds, new multiporphyrin archetypes that feature macrocycle-to-macrocycle electronic communication that stems from both π-cofacial and π-conjugative interactions.

Those skilled in the art will recognize the wide variety of cofacial macrocyclic assemblies can be prepared from the porphyrin-containing compounds of the invention. A number of assemblies are shown in the Experimental portion herein.

The monomeric macrocyclic building blocks can, for example, be porphyrins. Those in the art will recognize that porphyrins are derivatives of porphine, a conjugated cyclic structure of four pyrrole rings linked through their 2- and 5-positions by methine bridges. Porphyrins can bear up to 12 substituents at meso (i.e. α) and pyrrolic (i.e.,β) positions thereof. See, e.g., U.S. Pat. Nos. 5,371,199, 5,783,306, and 5,986,090 which are incorporated by reference. Porphyrins can be covalently attached to other molecules. The electronic features of the porphyrin ring system can be altered by the attachment of one or more substituents. The term “porphyrin” includes derivatives wherein a metal atom is inserted into the ring system, as well as molecular systems in which ligands are attached to the metal. The substituents, as well as the overall porphyrin structure, can be neutral, positively charged, or negatively charged.

A series of monomeric aryl- and perfluoroalkyl-functionalized (porphinato)zinc(II) units are employed as building blocks in these syntheses. The electronic properties of these two broad classes of (porphinato)zinc(II) compounds vary markedly; note, for example, that [5,10,15,20-tetrakis(perfluoroalkyl)porphinato]zinc(II) complexes possess HOMO and LUMO levels that are stabilized uniformly by nearly 700 mV relative to their corresponding (5,10,15,20-tetraphenylporphinato)zinc(II) benchmarks.

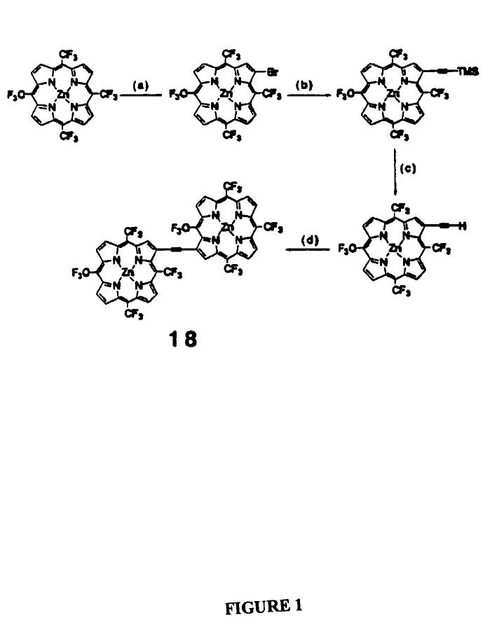

Macrocycle-to-macrocycle ethyne-bridged bis[(porphinato)metal] compounds serve as key precursors to cofacial porphyrin compounds synthesized via metal-templated cycloaddition reactions. The synthesis of the extremely electron-poor ethyne-linked dimeric (porphinato)zinc(II) species, bis[([(2,2′-5,10,15,20-trifluoromethyl]porphinato)zinc(II)]ethyne (18), is depicted in FIG. 1. In FIG. 1, the reagents and conditions are: (a) N-bromosuccinimide (1.1 equiv), methanol, reflux, 1.5 h (42%); (b) trimethylsilylacetylene (10 equiv), Pd(PPh₃)₂Cl₂ (10 mol %), CuI (10 mol %), THF/TEA 5:2, 40 C, 17 h (75%); (c) (i) TBAF (2 equiv), THF, 10 min, (ii) NaHCO₃ (aq) (96%); (d) 1 (1.2 equiv), Pd₂(dba)₃ (15 mol %), AsPh₃ (1.2 equiv), THF/TEA 5:1, 40 C, 17 h (73%). [(5,10,15,20-Trifluoromethyl)porphinato]zinc(II) can be readily brominated with N-bromosuccinimide, producing [(2-bromo-5,10,15,20-trifluoromethyl)porphinato]zinc(II); palladium-catalyzed cross-coupling with trimethylsilylacetylene gives the ethyne-elaborated macrocycle [5,10,15,20-(trifluoromethyl)-2-(trimethylsilylethynyl)porphinato]zinc(II). Deprotection with TBAF yields [(2-ethynyl-5,10,15,20-trifluoromethyl)porphinato]zinc(II), which can be cross-coupled with [(2-bromo-5,10,15,20-trifluoromethyl)porphinato]zinc(II) to afford 18. Compound 18 serves as the first example of an electron-deficient ethyne-bridged bis[(porphinato)metal] species; notably these reactions demonstrate that perfluoroalkyl-substituted porphyrin macrocycles can be both suitably derivatized and coupled in yields comparable to that established previously for their electron-rich counterparts.

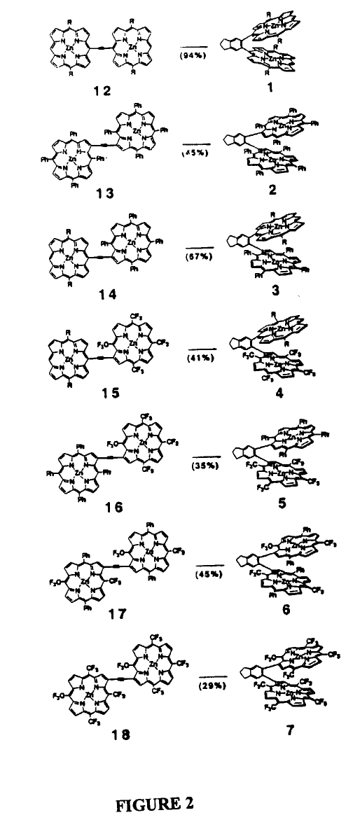

As such, ethyne-bridged precursor compounds bis[(5,5′-10,20-bis[4-(3 -methoxy-3-methylbutoxy)phenyl]porphinato)zinc(II)]ethyne (12), bis[(2,2′-5,10,15,20-tetraphenylporphinato)zinc(II)]ethyne (13), ([2-5,10,15,20-tetraphenylporphinato]zinc(II))-[5′-10′20′-bis[4-(3-methoxy-3-methylbutoxy)phenyl]porphinato)zinc(II)]ethyne (14), ([2-5,10,15,20-tetrakis(trifluoromethyl)porphinato]zinc(II))-[(5′-10′20′-bis[4-(3-methoxy-3-methylbutoxy)phenyl]porphinato)zinc(II)]ethyne (15), ([2-5,10,15,20-tetrakis(trifluoromethyl)porphinato]zinc(II))-[(2′-5′,10′,15′,20′-tetraphenylporphinato)zinc(II)]ethyne (16), and bis([2-5,15-(trifluoromethyl)-10,20-diphenylporphinato]zinc(II))ethyne (17) were all fabricated in a straightforward fashion. Ethyne-bridged, bis[(porphinato)zinc(II)] species 12-18 were converted to their corresponding 1,2-phenylene-bridged bis[(porphinato)metal compounds (FIG. 2) via a Co-templated cycloaddition with 1,6-heptadiyne; note that in these species (1-7), both macrocycle electronic structure and the (porphinato)metal-(porphinato)metal linkage topology are varied systematically. The general reaction conditions employed in FIG. 2 are (i) Co₂(CO)₈ (1 equiv), toluene/dioxane 5:1, 100 C, 10 min; (ii) 1,6-heptadiyne (20 equiv), Co₂(CO)₈ (1 equiv), 5 mL of toluene, added 17 h dropwise.

The [2+2+2] cycloaddition protocol was optimized for substrate 12, which was converted to 5,6-bis[(5′,5″-10′,20′-bis[4-(3-methoxy-3 -methylbutoxy)phenyl]porphinato)zinc(II)]indane (1) in 94% yield; these reaction conditions were employed for all ethyne-bridged porphyrin substrates in this study. It is well known that such metal-mediated cycloaddition transformations are sensitive to both alkyne steric and electronic properties; hence, the range of yields observed for this series is not surprising, given that reaction time was held essentially constant. Interestingly, in most cases, more than 90% of the porphyrin reactant was accounted for either as product or recovered starting material (see Experimental Section); as deleterious side reactions appear lacking, it is likely that each of these transformations can be optimized individually to give similarly high yields as that observed for the 12-to-1 conversion.

While 1,2-phenylene-bridged bis[(porphinato)metal] compounds analogous to meso-meso bridged 1 and meso-β bridged 5-([2′-5′,10′,15′,20′-tetraphenylporphinato]zinc(II))-6-[(5″-10″,20″-bis[4-(3-methoxy-3-methylbutoxy)phenyl]porphinato)zinc(II)]indane (3) have been reported, 5,6-bis[(2′-5′,10′,15′,20′-tetraphenylporphinato)zinc(II)]indane (2) and 5,6-bis([2′-5′,10′,15′,20′-tetrakis(trifluoromethyl)porphinato]zinc,(II))indane (7) serve as archetypal examples of β-β bridged cofacial porphyrin structures. Products 5-([2′-5′,10′,15′,20′-tetrakis(trifluoromethyl)porphinato]zinc(II))-6-[(5″-10″,20″-bis[4-(3 -methoxy-3-methylbutoxy)phenyl]porphinato)zinc(II)]indane (4), 5-(2′-5′,10′,15′,20′-[tetrakis(trifluoromethyl)porphinato]zinc(II))-6-[(2″-5″,10″,15″,20″-tetraphenylporphinato)zinc(II)]indane (5), 5,6-bis([2′-5′,15′-(trifluoromethyl)-10′,20′-diphenylporphinato]zinc(II)indane (6), and 7 are the first examples of cofacial bis[(porphinato)metal] complexes that possess perfluoroalkyl-substituted macrocycles; furthermore, these species highlight that this synthetic strategy enables extensive regulation of both the extent of macrocycle-macrocycle electronic asymmetry and the energetics of the cofacial (porphinato)metal frontier orbitals (vide infra).

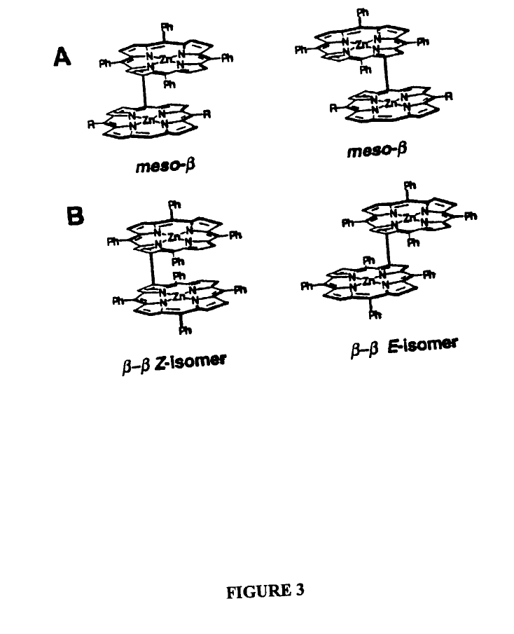

The nature of interporphyryl π-π interactions will play a pivotal role in determining the electronic properties of cofacial bis[(porphinato)metal] complexes and will be determined by the indane-to-porphyrin macrocycle linkage topology (FIG. 3). Unlike 1,2-phenylene-bridged cofacial porphyrin structures with meso-meso connectivity, two possible structural conformations can be manifest in meso-β and β-β bridged systems. In the meso- bridging motif highlighted in 3 and 4 (FIG. 3A), the two enantiomers possess equivalent π-cofacial electronic interactions. In contrast, disparate π-π interactions are evident for the two β-β bridged atropisomers possible for 2, 5, 6, and 7 (FIG. 3B). Both ¹H and ¹⁹F NMR analyses of each of these β-β bridged bis[(porphinato)zinc(II)] complexes confirm that only one atropisomer exists in solution at room temperature (see Experimental Section), presumably due to the fact that the eclipsed Z isomer possesses augmented steric strain relative to the E configuration (FIG. 3B).

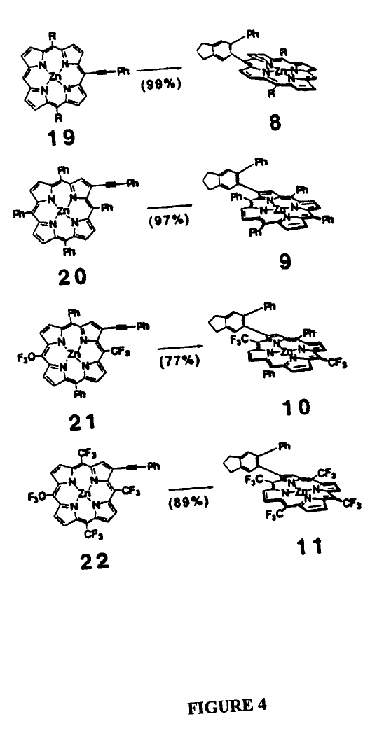

To facilitate the analyses of spectroscopic and potentiometric data for these cofacial bis[(porphinato)zinc(II)] complexes, an appropriate set of monomeric standards was synthesized. 5-(6-Phenyl)indanyl-derivatized porphyrin monomers (FIG. 4) were constructed via palladium-catalyzed cross-coupling of ethynylbenzene with meso- or β-brominated porphyrin precursors, followed by cycloaddition with 1,6-heptadiyne; these compounds (8-11, FIG. 4) were utilized as analytical benchmarks in lieu of simple 5,15- or 5,10,15,20-derivatized porphyrins. The general reaction conditions employed in FIG. 4 are (i) Co₂(CO)₈ (1 equiv), toluene/dioxane 5:1, 100 C, 10 min; (ii) 1,6-heptadiyne (10 equiv), 5 mL of toluene, added 90 min dropwise.

In one aspect, the invention concerns a facially-functionalized (porphinato)metal species. Reaction of I-III under the conditions described in Fogure 1 constitutes a powerful new route into cofacial porphyrin compounds. Since the first reports of these remarkable species, only minor methodological advancements have been made with respect to the conventional pyrrole and aldehyde condensation routes to rigidly linked cofacial porphyrin structures; this has both limited the range of electronic structural modifications possible in such constructs and required considerable synthetic effort to build related, rigid face-to-face structures that comprise more than two porphyrin units.

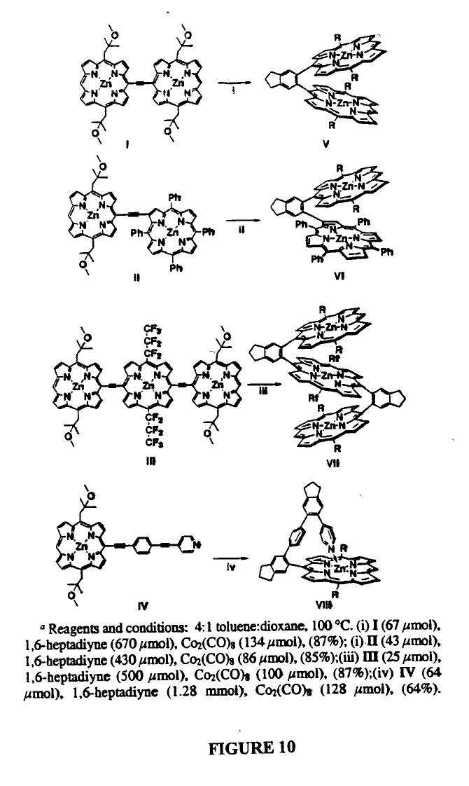

Meso-to-meso ethyne-bridged I serves as a precursor to V (FIG. 10), which features a ligand motif closely related to the 1,2-diporphyrylphenylene frameworks shown by Naruta to support metal-catalyzed homogeneous oxidation of water. Interestingly, Osuka, K.; Nakajima, S.; Nagata, T.; Maruyama, K.; Toriumi, K. Angew. Chem. Int. Ed. Engl. 1991, 30, 582-584 have reported the structure of 1,2-bis[5′-(15′-(p-tolyl)porphinatolzinc(II)]benzene, which features coplanar (porphinato)zinc(II) units that manifest a minimal 3.43 Å average interplanar separation distance.9a V-(MeOH)₂ thus constitutes an open-structure analogue of Osuka's 1,2-diporphyrylphenylene complex having a cavity-bound, hydrocarbon-based small molecule redox substrate. Key metrical parameters of V.(MeOH)₂ include a Zn-Zn distance of 6.54 Å, a large 56.4 dihedral angle between least-squares planes defined by the four central nitrogen atoms of its respective (porphinato)zinc(II) units, and a substantive lateral shift between the two porphyryl zinc atoms of 3.66 Å. Importantly, the V.(MeOH)₂ structure underscores: (i) that even when (porphinato)metal species are held in a cofacial arrangement by a 1,2-phenylene bridge, the structural plasticity necessary to accommodate both substrates and intermediates in catalytic redox cycles is clearly inherent, and (ii) that the energies required to distort a rigid, cofacial bis[(porphinato)metal] compound into the open “Pac-Man” structure 13 are no more than that provided by axial ligation and crystal packing forces.

The conversion of II to VI exemplifies the capacity of metal-templated cycloaddition reactions to assemble new classes of such structures, producing the first example of a covalently bridged, conformationally well-defined cofacial bis(porphyrin) system featuring meso-to-connectivity. While conventional face-to-face porphyrin structures that feature macrocycles linked to a rigid bridge via their respective meso-carbon positions display excitonic interactions in the B-band region when the interplanar separations are not unduly large, the oscillator strength of the low-energy B-state exciton component is modest due to its formally dipole forbidden nature. Because the x- and y-polarized B-states of a given (porphinato)zinc(II) unit in VI are rigorously precluded from being superimposable with the analogous x′- and y′-polarized states of the (porphinato)zinc(II) unit held cofacial to it, the low-energy exciton band displays dramatically enhanced intensity in comparison to meso-to-meso bridged bis(porphyrin) compounds.

The use of tris[(porphinato)zinc(II)] complex III as a starting material for these for these Co-assisted cycloadditions demonstrates that new cofacial porphyrin prototypes can be accessed in a straightforward manner and that substantial variation in macrocycle electronic structure does not limit the scope of such reactions. Compound VII is an example of a newly defined class of conjugated porphyrin arrays in which adjacent (porphinato)metal units differ substantially with respect to their electronic structure; potentiometric analysis shows that [5,15-di(perfluoroalkyl)porphinato]zinc(II) species possess HOMOs and LUMOs that are uniformly lowered in energy by ˜0.33 V relative to their conventional, electron-rich counterparts. Congruently, VII's optical spectrum shows clear evidence of charge resonance character in the prominent absorption bands, and photophysical behavior consistent with low-lying charge-transfer states.

Co-assisted cycloaddition of IV and 1,6-heptadiyne produces VIII, a complex that differs markedly from other previously delineated examples of (porphinato)metal species that feature covalently attached axial ligands. Because the metal-bound axial pyridyl moiety of VIII is linked directly to the macrocycle via an entirely aromatic structure, the conformation of the multidentate ligand is highly restricted. Such coordination environments built via two successive metal-templated cycloaddition reactions define a potential approach to control rigorously metal-centered redox potential and d orbital occupancy, as well as a synthetic strategy to be exploited in heme protein bioinorganic chemistry.

Metal-templated cycloaddition reactions involving appropriately elaborated porphyrinic synthons not only define new routes to facially functionalized (porphinato)metal species and cofacial porphyrin structures; they provide a new means to modulate optical and electronic properties within these structural motifs. With respect to this latter class of compounds, it is important to note that because palladium-catalyzed cross-coupling of ethyne- and halogen-bearing porphyrin templates makes straightforward the syntheses of heterobimetallic, electron-rich, electrophilic, and electronically asymmetric ethyne-bridged bis(porphyrin) systems, sequential cross-coupling/cycloaddition reactions enable the construction of entirely new classes of cofacial porphyrin species; such structures will make manifest numerous new opportunities with respect to redox catalyst design.

### EXPERIMENTAL

Certain aspects of the invention are illustrated by the following examples which are not intended to be limiting.

Inert atmosphere manipulations were carried out under nitrogen prepurified by passage through an O₂ scrubbing tower (Schweizerhall R3-11 catalyst) and a drying tower (Linde 3-Å molecular sieves). Air-sensitive solids were handled in a Braun 150-M glovebox. Standard Schlenk techniques were employed to manipulate air-sensitive solutions. A syringe pump was utilized to control reproducibly the time-dependent concentration of reagents in all metal-templated cycloaddition reactions.

Unless otherwise noted, all solvents utilized in this work were obtained from Fisher Scientific (HPLC grade) and distilled under nitrogen. Tetrahydrofuran and toluene were distilled from Na/benzophenone, and triethylamine was distilled from CaH₂; dioxane (anhydrous) was used as received from Aldrich. Pd₂dba₃, AsPh₃, and Co₂(CO)₈ were obtained from Strem. 1,6-Heptadiyne, TBAF, and ethynylbenzene were obtained from Aldrich. Trimethylsilylacetylene and triisopropylsilylacetylene were obtained from GFS Chemicals. Halogenated derivatives of [5,10,15,20-tetrakis(trifluoromethyl)porphinato]zinc(II) and [5,15-bis(trifluoromethyl)- 10,20-diphenylporphinato]zinc(II) species were prepared similarly to methods reported previously; see Fletcher, J. T.; Therien, M. J. J. Am. Chem. Soc. 2002, 124, 4298-4311 and Fletcher, J. T.; Therien, M. J. Inorganic Chemistry 2002, 41, 331-341.

Chromatographic purification (Silica 60, 230-400 mesh, EM Science) of all compounds was performed on the benchtop. Chemical shifts for 1H NMR spectra are relative to residual protium (CDCl₃, 7.24 ppm), while those for 19F NMR spectra are referenced to fluorotrichloromethane (=0.00 ppm).

Electronic spectra were recorded on an OLIS UV/visible/NIR spectrophotometry system that is based on the optics of a Carey 14 spectrophotometer. Cyclic voltammetric measurements were carried out with a PAR 273 electrochemical analyzer and a single-compartment electrochemical cell. ¹H and ¹⁹F NMR experiments were performed respectively on 250- and 200-MHz Bruker instruments.

MALDI-TOF mass spectroscopic data were obtained with a Perceptive Voyager DE instrument in the Laboratories of Dr. Virgil Percec (Department of Chemistry, University of Pennsylvania). Samples were prepared as micromolar solutions in THF, and dithranol (Aldrich) was utilized as the matrix. Cyclic voltammetric experiments were performed with an EG&G Princeton Applied Research model 273A potentiostat/galvanostat.

### EXAMPLE 1

5,15-Diphenylporphyrin

A flame-dried 1000 ml flask equipped with a magnetic stirring bar was charged with 2,2-dipyrrylmethane (458 mg, 3.1 mmol), benzaldehyde (315 μl, 3.1 mmol), and 600 ml of freshly distilled (CaH₂) methylene chloride. The solution was degassed with a stream of dry nitrogen for 10 minutes. Trifluoroacetic acid (150 μl, 1.95 mmol) was added via syringe, the flask was shielded from light with aluminum foil, and the solution was stirred for two hours at room temperature. The reaction was quenched by the addition of 2,3-dichloro-5,6-dicyano-1,4-benzoquinone (DDQ, 900 mg, 3.96 mmol) and the reaction was stirred for an additional 30 minutes. The reaction mixture was neutralized with 3 ml of triethylamine and poured directly onto a silica gel column (20×2 cm) packed in hexane. The product was eluted in 700 ml of solvent. The solvent was evaporated, leaving purple crystals (518 mg., 1.12 mmol, 72.2%). This product was sufficiently pure for further reactions. Vis(CHCl₃): 421 (5.55), 489 (3.63), 521 (4.20), 556 (4.04), 601 (3.71), 658 (3.73).

### EXAMPLE 2

5,15-Dibromo-10,20-Diphenylporphyrin

5,15-Diphenylporphyrin (518 mg, 1.12 mmol) was dissolved in 250 ml of chloroform and cooled to 0° C. Pyridine (0.5 ml) was added to act as an acid scavenger. N-Bromosuccinimide (400 mg, 2.2 mmol) was added directly to the flask and the mixture was followed by TLC (50% CH₂Cl₂/hexanes eluant). After 10 minutes the reaction reached completion and was quenched with 20 ml of acetone. The solvents were evaporated and the product was washed with several portions of methanol and pumped dry to yield 587 mg (0.94 mmol, 85%) of reddish-purple solid. The compound was sufficiently pure to use in the next reaction. Vis(CHCl₃): 421 (5.55), 489 (3.63), 521 (4.20), 556 (4.04), 601 (3.71), 658 (3.73).

### EXAMPLE 3

5,15-Dibromo-10,20-Diphenylporphyrinato Zinc

5,15-Dibromo-10,20-diphenylporphyrin (587 mg, 0.94 mmol) was suspended in 30 ml DMF containing 500 mg ZnCl₂. The mixture was heated at reflux for 2 hours and poured into distilled water. The precipitated purple solid was filtered through a fine fritted disk and washed with water, methanol, and acetone and dried in vacuo to yield 610 mg (0.89 mmol, 95%) of reddish purple solid. The compound was recrystallized from THF/heptane to yield large purple crystals of the title compound (564 mg, 0.82 mmol, 88%). Vis(THF): 428 (5.50), 526 (3.53), 541 (3.66), 564 (4.17), 606 (3.95).

### EXAMPLE 4

General Procedure for the Preparation of Ethyne-Bridged Bis[(Porphinato)Zinc(II)] Complexes

A 50-mL Schlenk tube was charged with a 2- or 5-ethynylporphyrin compound (1 equiv), a meso- or -bromoporphyrin complex (1.2 equiv), Pd₂(dba)₃ (0.15 equiv), and AsPh₃ (1.2 equiv). These reagents were dissolved in 5:1 THF/TEA and stirred for 3-26 h at 40 C. Following evaporation of the solvent, the residue was purified via chromatography. See generally Lin, V. S.-Y.; Therien, M. J. Chem. Eur. J 1995, 1, 645-651 and Lin, V. S.- Y.; DiMagno, S. G., Therien, M. J. Science 1994, 264, 1105-1111.

### EXAMPLE 5

Bis[(5,5′-10,20-Bis[4-(3-Methoxy-3-Methylbutoxy)Phenyl]Porphinato)Zinc(II)Ethyne (12).

Reagents: (5-bromo-10,20-bis[4-(3-methoxy-3-methylbutoxy)phenyl]porphinato)zinc(II) (53 mg, 0.068 mmol), (5-ethynyl-10,20-bis[4-(3-methoxy-3-methylbutoxy)phenyl]porphinato)zinc(II) (45 mg, 0.062 mmol), Pd₂(dba)₃ (9 mg, 0.0093 mmol), AsPh₃ (23 mg, 0.074 mmol), THF (10 mL), and triethylamine (2 mL). Reaction time, 3 h. Chromatographic purification: silica gel, 1:1 hexanes/THF, followed by SX-1 biobeads, THF. The dark green band was isolated, giving desired product 12 (0.083 g, 94% based on 45 mg of the porphyrinic starting material).

1H NMR (250 MHz, 50:1 CDCl₃/pyridine-d₅): 10.45 (d, J=4.53 Hz, 4H), 10.04 (s, 2H), 9.24 (d, J=4.43 Hz, 4H), 9.15 (d, J=4.43 Hz, 4H), 8.98 (d, J=4.35 Hz, 4H), 8.16 (d, J=8.43 Hz, 8H), 7.31 (d, J=8.53 Hz, 8H), 4.38 (t, J=7.26 Hz, 8H), 3.33 (s, 12H), 2.22 (t, J=6.98 Hz, 8H), 1.37 (s, 24H). Visible (THF): 403 (5.08), 411 (5.08), 430 (5.00), 478 (5.46), 548 (4.21), 565 (4.18), 701 (4.69) nm. MS (MALDI-TOF) m/z: 1535 (calcd for C₉₀H₈₆N₈O₈Zn₂ 1535).

### EXAMPLE 6

Bis[(2-5,10,15,20-Tetraphenylporphinato)Zinc(II)]Ethyne (13).

This compound was synthesized by methods reported previously (Lin, V. S.-Y.; Therien, M. J. Chem. Eur. J 1995, 1, 645-651).

### EXAMPLE 7

([2-5,10,15,20-Tetraphenylporphinato]Zinc(II))-[5′-10′20′-Bis[4-(3-Methoxy-3-Methylbutoxy)Phenyl]Porphinato)Zinc(II)Ethyne (14).

Reagents: (2-ethynyl-5,10,15,20-tetraphenylporphinato)zinc(II) (45 mg, 0.0641 mmol), (5-bromo-10,20-bis[4-(3-methoxy-3-methylbutoxy)phenyl]porphinato)zinc(II) (55 mg, 0.0705 mmol), Pd₂(dba)₃ (9 mg, 0.0096 mmol), AsPh₃ (24 mg, 0.0769 mmol), THF (10 mL), and triethylamine (2 mL). Reaction time, 26 h. Chromatographic purification: silica gel, 7:3 hexanes/THF. The green band was isolated, giving desired product 14 (51 mg, 57% based on 45 mg of the porphyrinic starting material).

1H NMR (250 MHz, 50:1 CDCl₃/pyridine-d₅): 10.04 (s, 1H), 9.61 (d, J=4.53 Hz, 2H), 9.50 (s, 1H), 9.24 (d, J=4.43 Hz, 2H), 8.97 (d, J=4.34 Hz, 2H), 8.91 (d, J=4.43 Hz, 2H), 8.88 (s, 2H), 8.83 (s, 2H), 8.77 (d, J=4.63 Hz, 1H), 8.72 (d, J=4.53 Hz, 1H), 8.27 (m, 4H), 8.18 (m, 4H), 8.13 (d, J=8.18 Hz, 4H), 7.67 (m, 9H), 7.27 (d, J=8.18 Hz, 4H), 6.90 (t, J=7.45 Hz, 2H), 5.67 (t, J=7.48 Hz, 1H), 4.35 (t, J=7.05 Hz, 4H), 3.31 (s, 6H), 2.19 (t, J=7.00 Hz, 4H), 1.34 (s, 12H). Visible (THF): 433 (5.37), 447 (5.46), 566 (4.41), 613 (4.34) nm. MS (MALDI-TOF) m/z: 1452 (calcd for C₉₀H₇₀N₈O₄Zn₂ 1454).

### EXAMPLE 8

([2-5,10,15,20-Tetrakis(Trifluoromethyl)Porphinato]Zinc(II))-[(5′-10′20′-Bis[4-(3-Methoxy-3-Methylbutoxy)Phenyl]Porphinato)Zinc(II)]Ethyne (15).

Reagents: (5-ethynyl- 10,20-bis[4-(3-methoxy-3-methylbutoxy)phenyl]porphinato)zinc(II) (40 mg, 0.055 mmol), [2-bromo-5,10,15,20-tetrakis(trifluoromethyl)porphinato]zinc(II) (48 mg, 0.066 mmol), Pd₂(dba)₃ (8 mg, 0.0083 mmol), AsPh₃ (20 mg, 0.066 mmol), THF (5 mL), and triethylamine (1 mL). Reaction time, 20 h. Chromatographic purification: silica gel, 3:1 hexanes/THF, followed by biobeads SX-1, THF. The dark green band was isolated, giving desired product 15 (65 mg, 86% based on 40 mg of the porphyrinic starting material).

1H NMR (250 MHz, 50:1 CDCl₃/pyridine-d₅): 10.22 (q, J=2.70 Hz, 1H), 10,21 (d, J=4.58 Hz, 2H), 10.09 (s, 1H), 9.65 (m, 6H), 9.26 (d, J=4.53 Hz, 2H), 9.15 (d, J=4.55 Hz, 2H), 8.99 (d, J=4.43 Hz, 2H), 8.15 (d, J=8.53 Hz, 4H), 7.30 (d, J=8.63 Hz, 4H), 4.39 (t, J=7.13 Hz, 4H), 3.34 (s, 6H), 2.23 (t, J=7.14 Hz, 4H), 1.38 (s, 12H). 19F NMR (200 MHz, 50:1 CDCl₃/pyridine-d₅): −33.74 (s, 3F), −36.42 (s, 3F), −36.74 (s, 3F), −36.83 (s, 3F). Visible (THF): 432 (5.24), 565 (4.16), 612 (4.13), 700 (4.12) nm. MS (MALDI-TOF) m/z: 1423 (calcd for C₇₀H₅₀F₁₂N₈O₄Zn₂ 1423).

### EXAMPLE 9

([2-5,10,15,20-Tetrakis(Trifluoromethyl)Porphinato]Zinc(II))-[(2′-5′,10′,15′,20′-Tetraphenylporphinato)Zinc(II)]Ethyne (16).

Reagents: (2-ethynyl-5,10,15,20-tetraphenylporphinato)zinc(II) (48 mg, 0.0684 mmol), [2-bromo-5,10,15,20-tetrakis(trifluoromethyl)porphinato]zinc(II) (50 mg, 0.0684 mmol), Pd₂(dba)₃ (9 mg, 0.0103 mmol), AsPh₃ (25 mg, 0.0821 mmol), THF (10 mL), and triethylamine (2 mL). Reaction time, 17 h. Chromatographic purification: silica gel, 4:1 hexanes/THF. The purple-green band was isolated, giving desired product 16 (47 mg, 51% based on 48 mg of the porphyrinic starting material).

1H NMR (250 MHz, 50:1 CDCl₃/pyridine-d₅): 9.65 (m, 7H), 9.38 (q, J=2.94 Hz, 1H), 8.93 (d, J=4.79 Hz, 1H), 8.86 (d, J=4.78 Hz, 1H), 8.83 (s, 2H), 8.80 (d, J=4.84 Hz, 1H), 8.75 (d, J=4.64 Hz, 1H), 8.36 (m, 4H), 8.19 (m, 4H), 7.85 (m, 3H), 7.73 (m, 9H). 19F NMR (200 MHz, 50:1 CDCl₃/pyridine-d₅): −34.24 (s, 3F), −36.51 (s, 3F), −36.83 (s, 3F), −37.10 (s, 3F). Visible (THF): 431 (5.39), 565 (4.37), 645 (4.47) nm. MS (MALDI-TOF) m/z: 1343 (calcd for C₇₀H₃₄F₁₂N₈Zn₂ 1342).

### EXAMPLE 10

Bis([2-5,15-(Trifluoromethyl)-10,20-Diphenylporphinato]Zinc(II))Ethyne (17).

Reagents: [2-bromo-5,15-bis(trifluoromethyl)-10,20-diphenylporphinato]zinc(II) (42 mg, 0.0612 mmol), [2-ethynyl-5,15-bis(trifluoromethyl)-10,20-diphenylporphinato]zinc(II) (49 mg, 0.0661 mmol), Pd₂dba₃ (8 mg, 0.0092 mmol), AsPh₃ (15 mg, 0.0735 mmol), THF (5 mL), and triethylamine (1 mL). Reaction time, 19 h. Chromatographic purification: silica gel, 1:1 hexanes/toluene. The final green band was isolated, giving desired product 17 (37 mg, 45% based on 42 mg of the (bromoporphinato)zinc(II) starting material).

1H NMR (250 MHz, 50:1 CDCl₃/pyridine-d₅): 9.70-9.40 (m, 6H), 8.90-8.70 (m, 8H), 8.30-7.90 (m, 8H), 7.90-7.40 (m, 12H). 19F NMR (200 MHz, 50:1 CDCl₃/pyridine-d₅): −32.45 (s, 3F), −35.74 (s, 3F). Visible (THF): 427 (5.21), 573 (4.17), 628 (sh) (4.54), 646 (4.66) nm. MS (MALDI-TOF) m/z: 1342 (calcd for C₇₀H₃₄F₁₂N₈Zn₂ 1342).

### EXAMPLE 11

Bis([2-5,10,15,20-Tetrakis(Trifluoromethyl)Porphinato]Zinc(II))Ethyne (18).

Reagents: [2-bromo-5,10,15,20-tetrakis(trifluoromethyl)porphinato]zinc(II) (61 mg, 0.0840 mmol), [2-ethynyl-5,10,15,20-tetrakis(trifluoromethyl)porphinato]zinc(II) (48 mg, 0.0700 mmol), Pd₂dba₃ (10 mg, 0.0105 mmol), AsPh₃ (26 mg, 0.0840 mmol), THF (10 mL), and triethylamine (2 mL). Reaction time, 17 h. Chromatographic purification: silica gel, 1:1 hexanes/toluene. The final green band was isolated, giving desired product 18 (67 mg, 73% based on 48 mg of the porphyrinic starting material).

1H NMR (50:1 CDCl₃/pyridine-d₅): 10.09 (q, J=2.78 Hz, 2H), 9.78 (m, 2H), 9.66 (m, 10H). 19F NMR (200 MHz, 50:1 CDCl₃/pyridine-d₅): −33.71 (s, 3F), −36.51 (s, 3F), −36.80 (s, 3F), −36.95 (s, 3F). Visible (THF): 425 (5.20), 572 (4.20), 620 (sh) (4.31), 655 (4.68) nm. MS (MALDI-TOF) m/z: 1311 (calcd for C₅₀H₁₄F₂₄N₈Zn₂ 1310).

### EXAMPLE 12

General Procedure for the Preparation of (Phenylethynylporphinato)Zinc(II) Complexes.

A 50-mL Schlenk tube was charged with ethynylbenzene (5-20 equiv), a meso- or β-bromoporphyrin (1 equiv), Pd₂(dba)₃ (0.15 equiv), and AsPh₃ (1.2 equiv). These reagents were dissolved in 5:1 THF/TEA and stirred for 14-20 h at 40 C. Following evaporation of the solvent, the residue was purified via chromatography. See LeCours, S. M.; Guan, H. -W.; DiMagno, S. G.; Wang, C. H.; Therien, M. J. J. Am. Chem. Soc. 1996, 118, 1497-1503 and LeCours, S. M.; DiMagno, S. G.; Therien, M. J. J. Am. Chem. Soc. 1996, 118, 11854-11864.

### EXAMPLE 13

(10,20-Bis[4-(3-Methoxy-3-Methylbutoxy)Phenyl]-5-(Phenylethynyl)Porphinato)Zinc(II) (19).

Reagents: (5-bromo-10,20-bis[4-(3-methoxy-3-methylbutoxy)phenyl]porphinato)zinc(II) (0.300 g, 0.384 mmol), ethynylbenzene (0.21 mL, 1.92 mmol), Pd₂(dba)₃ (0.053 g, 0.058 mmol),-AsPh₃ (0.141 g, 0.461 mmol), THF (15 mL), and triethylamine (3 mL). Reaction time, 14 h. Chromatographic purification: silica gel, 3:1 hexanes/THF. The dark green band was isolated, giving desired product 19 (0.270 g, 88% based on 300 mg of the (bromoporphinato)zinc(II) starting material).

1H NMR (250 MHz, 50:1 CDCl₃/pyridine-d₅): 10.04 (s, 1H), 9.77 (d, J=4.45 Hz, 2H), 9.22 (d, J=4.43 Hz, 2H), 8.98 (d, J=4.68 Hz, 2H), 8.93 (d, J=4.48 Hz, 2H), 8.07 (d, J=8.40 Hz, 4H), 8.00 (m, 2H), 7.49 (m, 2H), 7.43 (m, 1H), 7.26 (d, J=8.40 Hz, 4H), 4.36 (t, J=7.16 Hz, 4H), 3.32 (s, 6H), 2.20 (t, J=7.14 Hz, 4H), 1.36 (s, 12H). Visible (THF): 435 (5.57), 529 (3.55), 567 (4.19), 615 (4.26) nm. MS (MALDI-TOF) m/z: 856 (calcd for C₅₂H₄₉N₄Zn 856).

### EXAMPLE 14

[5,10,15,20-Tetraphenyl-2-(Phenylethynyl)Porphinato]Zinc(II) (20).

Reagents: (2-ethynyl-5,10,15,20-tetraphenylporphinato)zinc(II) (68 mg, 0.097 mmol), iodobenzene (0.11 mL, 0.97 mmol), Pd₂(dba)₃ (13 mg, 0.015 mmol), AsPh₃ (0.036 g, 0.116 mmol), THF (5 mL), and triethylamine (1 mL). Reaction time, 20 h. Chromatographic purification: silica gel, 4:1 hexanes/THF. The purple-green band was isolated, giving desired product 20 (59 mg, 78% based on 68 mg of the porphyrinic starting material).

1H NMR (250 MHz, 50:1 CDCl₃/pyridine-d₅): 9.14 (s, 1H), 8.81 (s, 2H), 8.80 (s, 2H), 8.76 (d, J=4.67 Hz, 1H), 8.67 (d, J=4.68 Hz, 1H), 8.15 (m, 8H), 7.67 (m, 14H), 7.35 (m, 3H). Visible (THF): 432, 524, 564, 600 nm. MS (MALDI-TOF) m/z: 776 (calcd for C₅₂H₃₂N₄Zn 776).

### EXAMPLE 15

[5,15-Bis(Trifluoromethyl)-10,20-Diphenyl-2-(Phenylethynyl)Porphinato]Zinc(II) (21).

Reagents: [2-bromo-5,15-bis(trifluoromethyl)-10,20-diphenylporphinatolzinc(II) (23 mg, 0.031 mmol), ethynylbenzene (34 L, 0.31 mmol), Pd₂dba₃ (4 mg, 0.0047 mmol), AsPh₃ (11 mg, 0.0372 mmol), THF (5 mL), and triethylamine (1 mL). Reaction time, 17 h. Chromatographic purification: silica gel, 1:1 hexanes/toluene. The green band was isolated, giving desired product 21 (18 mg, 76% based on 23 mg of the (bromoporphinato)zinc(II) starting material).

1H NMR (250 MHz, 50:1 CDCl₃/pyridine-d₅): 9.53 (m, 3H), 8.86 (s, 1H), 8.80 (d, J=4.98 Hz, 1H), 8.77 (d, J=4.71 Hz, 1H), 8.75 (d, J=4.0.82 Hz, 1H), 8.07 (m, 4H), 7.78 (m, 2H), 7.70 (m, 6H), 7.40 (m, 3H). 19F NMR (200 MHz, 50:1 CDCl₃/pyridine-d₅): −32.39 (s, 3F), −35.77 (s, 3F). Visible (THF): 430 (5.13), 571 (3.89), 615 (4.30) nm. MS (MALDI-TOF) m/z: 760 (calcd for C₄₂H₂₂F₆N₄Zn 760).

### EXAMPLE 16

[5,10,15,20-Tetrakis(Trifluoromethyl)-2-(Phenylethynyl)Porphinato]Zinc(II) (22).

Reagents: [2-bromo-5,10,15,20-tetrakis(trifluoromethyl)porphinato]zinc(II) (45 mg, 0.0621 mmol), ethynylbenzene (37 mL, 0.336 mmol), Pd₂dba₃ (9 mg, 0.0101 mmol), AsPh₃ (25 mg, 0.0806 mol), THF (5 mL), and triethylamine (1 mL). Reaction time, 20 h. Chromatographic purification: silica gel, 1:1 hexanes/toluene. The green band was isolated, giving desired product 22 (41 mg, 89% based on 45 mg of the (bromoporphinato)zinc(II) starting material).

1H NMR (50:1 CDCl₃/pyridine-d₅): 9.69 (q, J=2.86 Hz, 1H), 9.58 (m, 5H), 7.85 (m, 2H), 7.46 (m, 2H), 7.10 (m, 1H). 19F NMR (200 MHz, 50:1 CDCl₃/pyridine-d₅): −33.79 (s, 3F), −36.55 (s, 3F), −36.84 (s, 3F), −37.12 (s, 3F); visible (THF) 424 (5.03), 574 (3.98), 624 (4.36) nm. MS (MALDI-TOF) m/z: 743 (calc for C₃₂H₁₂F₁₂N₄Zn 744).

### EXAMPLE 17

Standard Procedure for the Syntheses of Cofacial Bis[(Porphinato)Zinc(II)] Compounds.

A 50-mL Schlenk tube was charged with an ethyne-bridged bis[(porphinato)zinc(II)] compound (1 equiv) and Co₂(CO)₈ (1 equiv). These reagents were dissolved in 5:1 toluene/dioxane and heated to 100 C; dropwise addition of 5 mL of a toluene solution containing 1,6-heptadiyne (20 equiv) and Co₂(CO)₈ (1 equiv) over a 17-h period followed. After the addition was complete, the solution was evaporated to dryness and the residue purified by chromatography. See Fletcher, J. T.; Therien, M. J. J. Am. Chem. Soc. 2000, 122, 12393-12394 and Fletcher, J. T.; Therien, M. J. J. Inorg. Chem. 2002, 41, 331-341.

### EXAMPLE 18

5,6-Bis[(5′,5″- 10′,20′-Bis[4-(3-Methoxy-3-Methylbutoxy)Phenyl]Porphinato)Zinc(II)]Indane (1).

Reagents: 12 (50 mg, 35 mol), Co₂(CO)₈ (12 mg, 35 mol), dioxane, (2.5 mL), and toluene (10 mL). Added dropwise: 1,6-heptadiyne (40 l, 350 mol) and Co₂(CO)₈ (12 mg, 35 mol). Chromatographic purification: silica gel, 3:2 hexanes/THF. The red band was isolated, giving desired product 1 (50 mg, 94% based on 50 mg of starting material 12).

1H NMR (250 MHz, pyridine-d₅): 10.31 (d, J=4.65 Hz, 4H), 10.11 (s, 2H), 9.29 (d, J=4.50 Hz, 4H), 9.13 (d, J=4.68 Hz, 4H), 8.95 (d, J=4.48 Hz, 4H), 8.64 (s, 2H), 8.02 (d, J=8.28 Hz, 4H), 7.66 (d, J=8.25 Hz, 4H), 7.32 (d, J=8.33 Hz, 4H), 7.25 (d, J=8.45 Hz, 4H), 4.43 (t, J=7.06 Hz, 8H), 3.41 (t, J=6.88 Hz, 4H), 3.30 (s, 12H), 2.40 (m, 2H), 2.26 (t, J=6.98 Hz, 8H), 1.36 (s, 24H). Visible (THF): 411 (5.54), 434 (4.74), 558 (4.32), 589 (3.72), 599 (3.71) nm. MS (MALDI-TOF) m/z: 1627 (calcd for C₉₇H₉₄N₈O₈Zn₂ 1627).

### EXAMPLE 19

5,6-Bis[(2′-5′,10′,15′,20′-Tetraphenylporphinato)Zinc(II)]Indane (2).

Reagents: 13 (25 mg, 18.1 mol), Co₂(CO)₈ (6 mg, 18.1 mol), dioxane (1 mL), and toluene (4 mL). Added dropwise: 1,6-heptadiyne (41 L, 362 mol) and Co₂(CO)₈ (6 mg, 18.1 mol). Chromatographic purification: silica gel, 1:1 hexanes/toluene. The purple band was isolated, giving desired product 2 (12 mg, 45% based on 25 mg of starting material 13).

1H NMR (250 MHz, 50:1 CDCl₃/pyridine-d₅): 8.46-8.92 (m, 14H), 6.80-8.42 (m, 42 H), 2.96 (d, J=7.2 Hz, 2H), 2.83 (t, J=7.2 Hz, 2H), 2.10 (m, 2H). Visible (THF): 424 (5.44), 561 (4.34), 599 (3.92) nm. MS (MALDI-TOF) m/z: 1468 (calcd for C₉₇H₆₂N₈Zn₂ 1466).

### EXAMPLE 20

5-([2′-5′,10′,15′,20′-Tetraphenylporphinato]Zinc(II))-6-[(5″-10″,20″-Bis[4-(3-Methoxy-3-Methylbutoxy)Phenyl]Porphinato)Zinc(II)Indane (3).

Reagents: 14 (25 mg, 17.8 mol), Co₂(CO)₈ (6 mg, 17.9 mol), dioxane (1 mL), and toluene (5 mL). Added dropwise: 1,6-heptadiyne (41 L, 357 mol) and Co₂(CO)₈ (6 mg, 17.9 mol). Chromatographic purification: silica gel, 4:1 hexanes/THF. The first green band was isolated, giving desired product 3 (18 mg, 67% based on 25 mg of starting material 14).

1H NMR (250 MHz, 50:1 CDCl₃/pyridine-d₅): 9.86 (s, 1H), 9.52 (d, J=4.61 Hz, 1H), 9.23 (d, J=4.51 Hz, 1H), 9.06 (d, J=4.37 Hz, 1H), 8.97 (d, J=4.38 Hz, 1H), 8.96 (d, J=4.25 Hz, 1H), 8.92 (d, J=4.57 Hz, 1H), 8.75 (m, 2H), 8.61 (m, 6H), 8.49 (d, J=4.70 Hz, 1H), 8.32 (d, J=4.68 Hz, 1H), 8.09 (m, 1H), 7.98 (m, 3H), 7.86 (m, 3H), 7.58 (m, 16H), 7.25 (m, 2H), 7.14 (m, 2H), 7.03 (m, 1H), 6.87 (m, 1H), 6.36 (m, 1H), 4.54 (m, 1H), 4.46 (t, J=7.18 Hz, 2H), 4.17 (t, J=7.15 Hz, 2H), 3.37 (s, 3H), 3.23 (s, 3H), 3.04 (t, J=6.80 Hz, 4H), 2.28 (t, J=7.09 Hz, 4H), 2.07 (t, J=7.12 Hz, 2H), 1.42 (s, 6H), 1.26 (s, 6H). Visible (THF): 418 (5.45), 438 (5.09), 555 (4.31), 595 (3.76) nm. MS (MALDI-TOF) m/z: 1546 (calcd for C₉₇H₇₈N₈O₄Zn₂ 1546). The final green band was also collected, which corresponded to pure starting material 14 (8 mg, 32%).

### EXAMPLE 21

5-([2′-5′,10′,15′,20′-Tetrakis(Trifluoromethyl)Porphinato]Zinc(II))-6-[(5″-10″,20″-Bis[4-(3-Methoxy-3-Methylbutoxy)Phenyl]Porphinato)Zinc(II)]Indane (4).

Reagents: 15 (25 mg, 18.2 mol), Co₂(CO)₈ (6 mg, 18.2 mol), dioxane (1 mL), and toluene (5 mL). Added dropwise: 1,6-heptadiyne (21 L, 182 mol) and Co₂(CO)₈ (6 mg, 18.2 mol). Chromatographic purification: silica gel, 4:1 hexanes/THF. The first green band was isolated, giving desired product 4 (11 mg, 41% based on 25 mg of starting material 15).

1H NMR (250 MHz, 50:1 CDCl₃/pyridine-d₅): 9.95 (s, 1H), 9.52 (s, 2H), 9.41 (s, 3H), 9.27 (d, J=4.53 Hz, 1H), 9.19 (m, 1H), 9.16 (d, J=4.73 Hz, 1H), 9.10 (d, J=4.43 Hz, 1H), 9.05 (d, J=4.45 Hz, 1H), 9.04 (d, J=4.48 Hz, 1H), 8.92 (m, 1H), 8.81 (d, J=4.55 Hz, 1H) 8.79 (m, 1H), 8.76 (d, J=4.43 Hz, 1H), 8.42 (s, 1H), 8.38 (dd, J1=8.27 Hz, J2=2.05 Hz, 1H), 8.07 (dd, J1=8.27 Hz, J2=1.96 Hz, 1H), 7.87 (dd, J1=8.27 Hz, J2=2.00 Hz, 1H), 7.81 (dd, J1=8.53 Hz, J2=2.00 Hz, 1H), 7.43 (dd, J1=8.49 Hz, J2=2.20 Hz, 1H), 7.28 (dd, J1=11.41 Hz, J2=3.16 Hz, 1H), 7.15 (dd, J1=10.8 Hz, J2=2.45 Hz, 1H), 7.09 (dd, J1=10.8 Hz, J2=2.45 Hz, 1H), 4.41 (t, J=7.13 Hz, 2H), 4.28 (t, J=7.18 Hz, 2H), 3.45 (m, 4H), 3.33 (s, 3H), 3.27 (s, 3H), 2.48 (m, 2H), 2.24 (t, J=7.15 Hz, 2H), 2.14 (t, J=7.15 Hz, 2H), 1.38 (s, 6H), 1.31 (s, 6H). 19F NMR (200 MHz, 50:1 CDCl₃/pyridine-d₅): −31.50 (s, 3F), −36.51 (s, 3F), −36.72 (s, 3F), −38.36 (s, 3F). Visible (THF): 418 (5.43), 555 (4.26), 612 (4.22) nm. MS (MALDI-TOF) m/z: 1514 (calcd for C₇₇H₅₈F₁₂N₈O₄Zn₂ 1514). The final green-brown band was also isolated, giving pure recovered starting material 15 (14 mg, 55%).

### EXAMPLE 22

5-(2′-5′,10′,15′,20′-[Tetrakis(Trifluoromethyl)Porphinato]Zinc(II))-6-[(2″-5″,10″,15″,20″-Tetraphenylporphinato)Zinc(II)]Indane (5).

Reagents: 16 (35 mg, 26 mol), Co₂(CO)₈ (9 mg, 26 mol), dioxane (1 mL), and toluene (5 mL). Added dropwise: 1,6-heptadiyne (30 L, 260 mol) and Co₂(CO)₈ (9 mg, 26 mol). Chromatographic purification: silica gel, 4:1 hexanes/THF. The first green band was isolated, giving desired product 5 (13 mg, 35% based on 35 mg of starting material 16).

1H NMR (250 MHz, 50:1 CDCl₃/pyridine-d₅): 9.74 (m, 1H), 9.63 (m, 1H), 9.57 (m, 2H), 9.37 (m, 1H), 9.13 (m, 1H), 9.04 (m, 1H), 8.93 (s, 1H), 8.90 (d, J=4.71 Hz, 1H), 8.88 (d, J=4.68 Hz, 1H), 8.86 (d, J=4.66 Hz, 1H), 8.82 (d, J=4.60 Hz, 1H), 8.74 (d, J=4.60 Hz, 1H), 8.65 (d, J=4.66 Hz, 1H), 8.44 (m, 1H), 8.30 (m, 1H), 8.23 (m, 4H), 8.16 (m, 4H), 7.70 (m, 8H), 7.10 (m, 2H), 6.64 (m, 2H), 2.83 (m, 4H), 2.08 (m, 2H). 19F NMR (200 MHz, 50:1 CDCl₃/pyridine-d₅): −32.79 (s, 3F), −36.07 (s, 3F), −36.36 (s, 3F), −38.19 (s, 3F). Visible (THF): 426 (5.36), 562 (4.83), 612 (4.22) nm. MS (MALDI-TOF) m/z: 1436 (calcd for C₇₇H₄₂F₁₂N₈Zn₂ 1434). A slower brown-green band, following the product band, was also isolated, giving pure recovered starting material 16 (21 mg, 60%).

### EXAMPLE 23

5,6-Bis([2′-5′,15′-(Trifluoromethyl)-10′,20′-Diphenylporphinato]Zinc(II))Indane (6).

Reagents: 17 (22 mg, 16.7 mol), Co₂(CO)₈ (6.5 mg, 18.6 mol), dioxane (1 mL), and toluene (4 mL). Added dropwise: 1,6-heptadiyne (25 L, 186 mol) and Co₂(CO)₈ (6.5 mg, 18.6 mol). Chromatographic purification: silica gel, 100:100:1 hexanes/toluene/THF. The green band was isolated, giving desired product 6 (12 mg, 45% based on 22 mg of starting material 17).

1H NMR (250 MHz, 50:1 CDCl₃/pyridine-d₅): 9.67 (m, 2H), 9.49 (m, 2H), 9.42 (m, 2H), 8.95-8.60 (m, 8H), 8.42 (s, 2H), 8.10-7.40 (m, 18H), 6.48 (m, 2H), 3.05 (m, 4H), 2.15 (m, 2H). 19F NMR (200 MHz, 50:1 CDCl₃/pyridine-d₅): −31.00 (s, 3F), −35.68 (s, 3F). Visible (THF): 419 (5.30), 564 (4.12), 610 (4.41) nm. MS (MALDI-TOF) m/z: 1434 (calcd for C₇₇H₄₂F₁₂N₈Zn₂ 1434).

### EXAMPLE 24

5,6-Bis([2′-5′,10′,15′,20′-Tetrakis(Trifluoromethyl)Porphinato]Zinc(II))Indane (7).

Reagents: 18 (16 mg, 12.2 mol), dioxane (1 mL), and toluene (4 mL). Added dropwise: 1,6-heptadiyne (28 L, 243 mol) and Co₂(CO)₈ (8 mg, 24.3 mol). Chromatographic purification: silica gel, 1:1 hexanes/toluene. The green band was isolated, giving desired product 7 (6 mg, 29% based on 16 mg of starting material 18).

1H NMR (250 MHz, 50:1 CDCl₃/pyridine-d₅): 9.65 (m, 2H), 9.53 (m, 4H), 9.12 (dq, J1=2.22 Hz, J2=3.01 Hz, 2H), 8.67 (dq, J1=2.60 Hz, J2=2.63 Hz, 2H), 8.48 (m, 2H), 8.35 (s, 2H), 8.18 (dq, J1=2.50 Hz, J2=2.67 Hz, 2H), 3.45 (m, 4H), 2.51 (m, 2H). 19F NMR (200 MHz, 50:1 CDCl₃/pyridine-d₅): −34.29 (s, 3F), −36.23 (s, 3F), −36.45 (s, 3F), −37.16 (s, 3F). Visible (THF): 399 (5.18), 570 (3.94), 608 (4.23) nm. MS (MALDI-TOF) m/z: 1400 (calcd for C₅₇H₂₂F₂₄N₈Zn₂ 1402). The final green band was also isolated, giving pure recovered starting material 18 (14 mg, 63%).

### EXAMPLE 25

Standard Procedure for Metal-Templated Cycloaddition Reactions Involving (Phenylethynylporphinato)Zinc(II) Substrates.

A 50-mL Schlenk tube was charged with a (phenylethynylporphinato)zinc(II) compound (1 equiv) and Co₂(CO)₈ (1 equiv). These reagents were dissolved in 5:1 toluene/dioxane and heated to 100 C; dropwise addition of 5 mL of a toluene solution containing 1,6-heptadiyne (10 eq) over a 90-min period followed. After the addition was complete, the solution was evaporated to dryness and the residue purified by chromatography. See Fletcher, J. T.; Therien, M. J. J. Am. Chem. Soc. 2000, 122, 12393-12394.

### EXAMPLE 26

5-Phenyl-6-[(5′-10′,20′-Bis[4-(3-Methoxy-3-Methylbutoxy)Phenyl]Porphinato)Zinc(II)]Indane (8).

Reagents: 19 (40 mg, 46.6 mol), Co₂(CO)₈ (16 mg, 46.6 mol), dioxane (1 mL),and toluene (4 mL). Added dropwise: 1,6-heptadiyne (53 L, 466 mol). Chromatographic purification: silica gel, 4:1 hexanes/THF. The purple band was isolated, giving desired product 8 (44 mg, 99% based on 40 mg of starting material 19).

1H NMR (250 MHz, 50:1 CDCl₃/pyridine-d₅): 10.11 (s, 1H), 9.21 (d, J=4.48 Hz, 2H), 8.93 (d, J=4.43 Hz, 2H), 8.87 (d, J=4.60 Hz, 2H), 8.81 (d, J=4.55 Hz, 2H), 8.03 (m, 4H), 7.81 (s, 1H), 7.57 (s, 1H), 7.20 (m, 4H), 7.08 (m, 2H), 6.24 (m, 3H), 4.32 (t, J=7.37 Hz, 4H), 3.30 (s, 6H), 3.24 (t, J=7.34 Hz, 2H), 3.07 (t, J=7.32 Hz, 2H), 2.30 (m, 2H), 2.18 (t, J=7.14 Hz, 4H), 1.34 (s, 12H). Visible (THF): 422 (5.56), 552 (4.21), 592 (3.57) nm. MS (MALDI-TOF) m/z: 948 (calcd for C₅₉H₅₆N₄O₄Zn 948).

### EXAMPLE 27

5-Phenyl-6-[(2′-5′,10′,15′,20′-Tetraphenylporphinato)Zinc(II)]Indane (9).

Reagents: 20 (59 mg, 75.8 mol), Co₂(CO)₈ (26 mg, 75.8 mol), dioxane (2 mL), and toluene (8 mL). Added dropwise: 1,6-heptadiyne (86 L, 758 mol). Chromatographic purification: silica gel, 4:1 hexanes/THF. The purple band was isolated, giving desired product 9 (64 mg, 97% based on 59 mg of starting material 20).

1H NMR (250 MHz, 50:1 CDCl₃/pyridine- d5): 8.83 (s, 2H), 8.81 (s, 1H), 8.80 (s, 1H), 8.72 (d, J=4.71 Hz, 1H), 8.58 (d, J=4.69 Hz, 1H), 8.45 (s, 1H), 8.15 (m, 6H), 7.90-7.10 (m,18H), 7.06 (m, 2H), 6.75 (m, 1H), 6.65 (m, 2H), 2.93 (t, J=7.25 Hz, 2H), 2.78 (t, J=7.33 Hz, 2H), 2.10 (m, 2H). Visible (THF): 427 (5.55), 558 (4.16), 597 (3.92) nm. MS (MALDI-TOF) m/z: 868 (calcd for C₅₉H₄₀N₄Zn 868).

### EXAMPLE 28

5-Phenyl-6-([2′-5′,15′-Bis(Trifluoromethyl)-10′,20′-Diphenylporphinato]Zinc(II))Indane (10).

Reagents: 21 (30 mg, 39.4 mol), Co₂(CO)₈ (13 mg, 26.8 mol), dioxane (1 mL), and toluene (5 mL). Added dropwise: 1,6-heptadiyne (45 L, 394 mol). Chromatographic purification: silica gel, 1:1 hexanes/toluene. The green band was isolated, giving desired product 10 (26 mg, 77% based on 30 mg of starting material 21).

1H NMR (250 MHz, 50:1 CDCl₃/pyridine-d₅): 9.54 (m, 3H), 8.85 (d, J=4.83 Hz, 1H), 8.83 (d, J=4.79 Hz, 1H), 8.79 (d, J=4.98 Hz, 1H), 8.39 (m, 1H), 8.02 (s, 1H), 7.88-7.42 (m, 13H), 7.10 (m, 3H), 3.12 (m, 2H), 2.98 (m, 2H), 2.20 (m,2H). 19F NMR (200 MHz, 50:1 CDCl₃/pyridine-d₅): −31.22 (s, 3F), −35.65 (s, 3F). Visible (THF): 424 (5.24), 566 (3.94), 608 (4.24) nm. MS (MALDI-TOF) m/z: 853 (calcd for C₄₉H₃₀F₆N₄Zn 852).

### EXAMPLE 29

5-Phenyl-6-([2′-5′,10′,15′,20′-Tetrakis(Trifluoromethyl)Porphinato]Zinc(II))Indane (11).

Reagents: 22 (20 mg, 26.8 mol), Co₂(CO)₈ (9 mg, 26.8 mol), dioxane (1 mL), and toluene (4 mL). Added dropwise: 1,6-heptadiyne (31 L, 268 mol). Chromatographic purification: silica gel, 1:1 hexanes/toluene. The green band was isolated, giving desired product 11 (20 mg, 89% based on 20 mg of starting material 22).

1H NMR (250 MHz, 50:1 CDCl₃/pyridine-d₅): 9.61 (m, 5H), 9.40 (dq, J1=2.51 Hz, J2=2.70 Hz, 1H), 9.01 (q, J=3.07 Hz, 1H), 7.72 (s, 1H), 7.51 (s, 1H), 7.07 (m, 2H), 6.52 (m, 3H), 3.15 (m, 4H), 2.27 (m, 2H). 19F NMR (200 MHz, 50:1 CDCl₃/pyridine-d₅): −33.18 (s, 3F), −36.15 (s, 3F), −36.57 (s, 3F), −37.21 (s, 3F). Visible (THF): 420 (5.10), 570 (3.96), 612 (4.22) nm. MS (MALDI-TOF) m/z: 837 (calcd for C₃₉H₂₀F₁₂N₄Zn 836).

### EXAMPLE 30

### Electronic Spectroscopy.

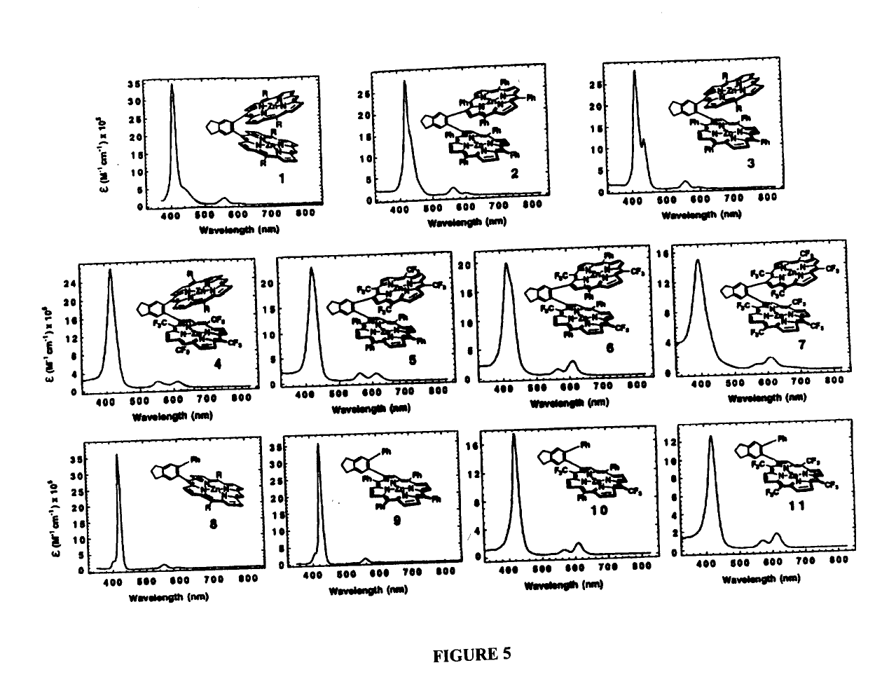

FIG. 5 depicts the electronic absorption spectra of cofacial bis[(porphinato)zinc(II)] complexes 1-7; absorption and emission data for these species are summarized in Table 1. A strongly allowed exciton band, blue-shifted with respect the Soret band of the simple (porphinato)zinc(II) building block, is a spectral hallmark of cofacial bis[porphyrin] compounds having modest porphyrin-porphyrin interplanar separations. The max of the Soret band of meso-meso bridged dimer 1 is shifted hypsochromically 634 cm-⁻¹ in comparison to benchmark 8. In contrast, β-β bridged dimer 2 is blue-shifted by only 166 cm-⁻¹ versus benchmark 9. Note, however, that electron-poor β-β bridged 7 displays strong exciton coupling, with the max of its Soret band hypsochromically shifted by 1253 cm-⁻¹ relative to 11. These data indicate that the relative degree of exciton coupling between porphyrin units is not dependent solely upon macrocycle-macrocycle relative orientation; the nature of the macrocycle frontier orbitals clearly plays a role as well (vide infra). The general electronic spectral features peculiar to cofacial [(porphinato)zinc(II)] complexes possessing either asymmetric connectivity or electronically disparate macrocycle units have been discussed in Fletcher, J. T.; Therien M. J. J. Am. Chem. Soc. 2000, 122, 12393-12394.

TABLE 1
Prominent Absorption and Emission Bands of 5,6-
Bis[(porphinato)zinc(II)]indane and 5-Phenyl-6-
[(porphinato)Zinc(II)]indane Complexes, Recorded in
THF Solvent electronic absorption

| | B-band region | | Q-band region | | | fluorescent emission | | |
|---|---|---|---|---|---|---|---|---|
| | λ | ν | log | λ | ν | log | λ | ν |
| Compd | (nm) | (cm−1) | (ε) | (nm) | (cm−1) | (ε) | (nm) | (cm−1) |
| 1 | 411 | 24.331 | 5.54 | 558 | 17.921 | 4.32 | 611 | 16.367 |
| | 434 | 23.041 | 4.74 | 589 | 16.978 | 3.72 | 657 | 15.521 |
| | | | | 599 | 16.695 | 3.71 | | |
| 2 | 424 | 23.585 | 5.44 | 561 | 17.825 | 4.34 | 617 | 16.207 |
| | | | | 599 | 16.694 | 3.92 | 662 | 15.106 |
| 3 | 418 | 23.923 | 5.45 | 555 | 18.018 | 4.31 | 612 | 16.340 |
| | 438 | 22.831 | 5.09 | 595 | 16.807 | 3.76 | 655 | 15.267 |
| 4 | 418 | 23.923 | 5.43 | 555 | 18.018 | 4.26 | 639 | 15.649 |
| | | | | 612 | 16.340 | 4.22 | 684 | 14.620 |
| 5 | 426 | 23.474 | 5.36 | 562 | 17.794 | 4.83 | 629 | 15.898 |
| | | | | 612 | 16.340 | 4.22 | | |
| 6 | 419 | 23.866 | 5.13 | 564 | 17.730 | 4.12 | 625 | 16.000 |
| | | | | 610 | 16.393 | 4.41 | | |
| 7 | 399 | 25.063 | 5.18 | 570 | 17.544 | 3.94 | 633 | 15.798 |
| | | | | 608 | 16.447 | 4.23 | | |
| 8 | 422 | 23.697 | 5.56 | 552 | 18.116 | 4.21 | 601 | 16.639 |
| | | | | 592 | 16.892 | 3.57 | 649 | 15.408 |
| 9 | 427 | 23.419 | 5.55 | 558 | 17.921 | 4.16 | 611 | 16.367 |
| | | | | 597 | 16.750 | 3.92 | 655 | 15.267 |
| 10 | 424 | 23.585 | 5.24 | 566 | 17.668 | 3.94 | 635 | 15.748 |
| | | | | 608 | 16.447 | 4.24 | | |
| 11 | 420 | 23.810 | 5.10 | 570 | 17.544 | 3.96 | 635 | 15.748 |
| | | | | 612 | 16.340 | 4.22 | | |

### EXAMPLE 31

Electrochemical Studies

A large number of dimeric porphyrin and (porphinato)zinc compounds bridged cofacially by rigid aryl spacers have been reported and their electronic properties relative to simple porphyrinic monomers have been discussed. Each of these previously reported systems were connected via the macrocycle meso positions, and the observed frontier orbital destabilization evident in the cofacial bis[(porphinato)metal] complex relative to the corresponding monomeric chromophore was attributed to electrostatic repulsions between the π-systems of the closely stacked porphyrin macrocycles. Due to the symmetry of the porphyrin macrocycle's frontier molecular orbitals, differences in both macrocycle-macrocycle connectivity and electronic structure should have a profound impact on the nature of electronic interactions in such-stacked structures. Potentiometric studies of the series of cofacial structures reported herein enable quantitative assessment of the extent to which these factors impact the frontier orbital energy levels of face-to-face bis[(porphinato)metal] compounds.

Structural studies evince that the cofacial orientation of 1,2-phenylene-bridged bis[(porphinato)metal] compounds is dependent strongly upon the solvent system used to obtain X-ray-quality crystals. When noncoordinating solvents are used, such structures adopt largely coplanar conformations; in contrast, when coordinating solvents are utilized, X-ray crystallographic studies show that cofacial bis[(porphinato)metal] species express open conformations possessing large dihedral angles between the porphyrin least-squares planes. Because coordinating solvents were utilized in our electrochemical studies, we consider only the open cofacial conformation in the analyses of these data.

In a 1,2-phenylene-bridged bis[(porphinato)zinc(II)] structure, a number of sub-van der Waals contacts are enforced, assuming a typical dihedral angle of 60 between macrocycle least-squares planes, as demonstrated by both computational studies and X-ray crystallographic analyses. For example, computational modeling indicates the closest of these cofacial contacts for a meso-meso bridged structure are at the C3, C5, and C7 positions of the macrocycles, which possess average interplanar atom-atom separations (C3-C3′, C5-C5′, C7-C7′) of less than 3.0 Å. Similar sub-van der Waals contacts are evident in the meso-β, and β-β structures (meso-β, C3-C5′, C5-C3′; β-β, C2-C4′, C4-C2′). Note also that each cofacial bis(porphyrin) linkage topology shows additional macrocycle-macrocycle atom-atom contacts at the van der Waals separation distance of ˜3.4 Å (meso-meso, C4-C4′, C6-C6′; meso-β, C4-C4′, C6-C2′; β-β, C3-C3′). Other porphyryl atomic positions manifest atom-atom interplanar separations exceeding -contact distances. For example, at a 60 dihedral angle between the macrocycle least-squares planes, the average N1-N1′ and N2-N2′ separations for the meso-meso linked structure are ˜4.9 Å; thus, our first-order analysis of the electronic interactions that fix the frontier orbital energy levels of 5,6-indanyl-bridged cofacial (porphinato)zinc(II) structures focus on these positions of close contact between the two macrocycle planes.

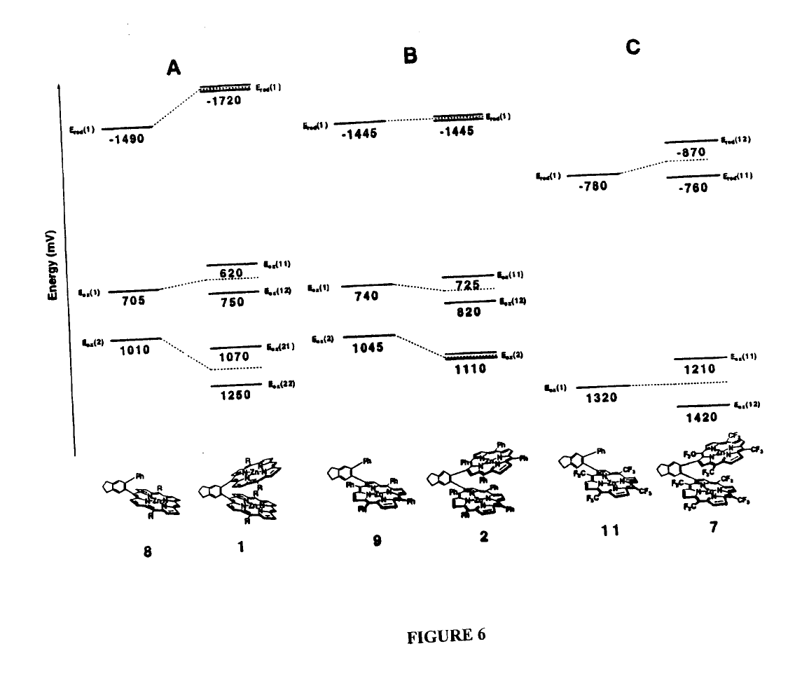

Cyclic voltammetric data for the 5,6-bis[(porphinato)zinc(II)]indane and 5-phenyl-6-[(porphinato)zinc(II)]indane complexes are presented in Table 2. It is evident from the comparison of the electron-rich bis[(porphinato)zinc(II)] species 1, 2, and 3 that macrocycle-macrocycle connectivity influences frontier orbital energy levels. Compound 1 (FIG. 6A) displays oxidative and reductive cyclic voltammetric processes consistent with those reported for meso-meso bridged cofacial (porphinato)zinc(II) dimers. Note that the initial anodic step of the first oxidative process of 1 is destabilized by 85 mV relative to monomeric 8, while the second step of the first oxidative process is stabilized by 45 mV relative to this benchmark (Table 2). These observed potentiometric shifts with respect to the (porphinato)zinc(II) monomer have been commonly attributed to electrostatically driven destabilization of the HOMO in the neutral bis[(porphinato)zinc(II)] complex and the fact that the cation radical state of the dimer gains added stability due to electronic delocalization. Consistent with previously reported electrochemical analyses, 1's initial cathodic redox process is shifted substantially (−230 mV) to more negative potential relative to reference monomer 8.

TABLE 2
Comparative Cyclic Voltammetric Data of the 5,6-
Bis[(porphinato)zinc(II)]indane Complexes and
5-Phenyl-6-[(porphinato)zinc(II)]indane Benchmarks.

| | E_{1/2} (mV) | | | |
|---|---|---|---|---|
| Compd | ZnP/ZNP⁺ | ZNP⁺/Zn²⁺ | ZnP/Zn²⁺ | ZnP⁻/ZnP |
| 1 | 620, 750 | 1070, 1250 | | −1720^{b} |
| 2 | 725, 820 | 1110^{b} | | −1445^{b} |
| 3 | 735, 805 | 1080, 1250 | | −1740^{b} |
| 4 | 710 | 990 | 1.420^{b} | −1070, −1650 |
| 5 | 740 | 995 | 1415^{b} | −980, −1455 |
| 6 | | | 1085^{b}, 1250^{b} | −1040, −1290 |
| 7 | | | 12110^{b}, 1420^{b} | −775, −890 |
| 8 | 705 | 1010 | | −1490 |
| 9 | 740 | 1045 | | −1445 |
| 10 | | 1090^{b} | | −985 |
| 11 | | 1320^{b} | | −780 |

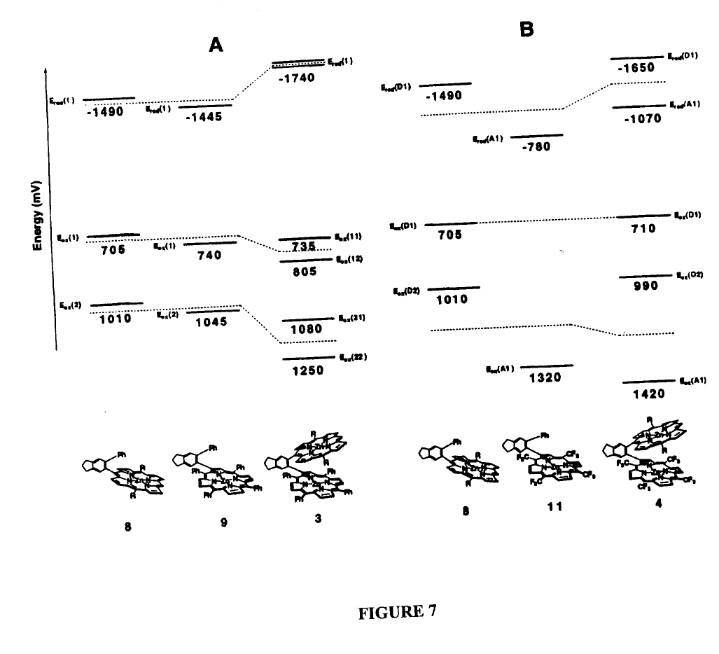

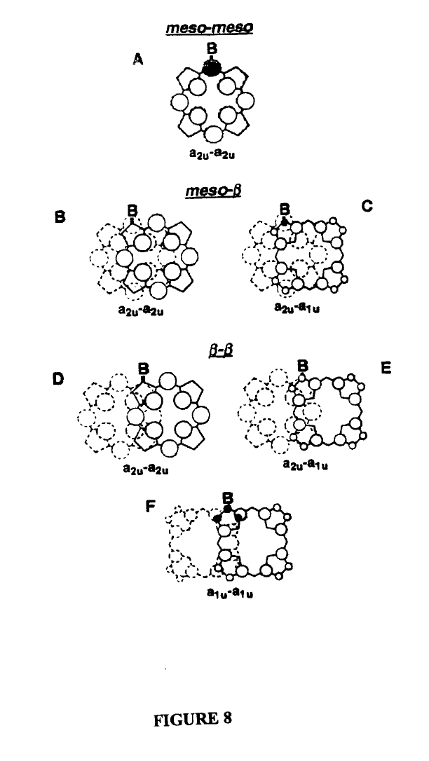

^{a}Experimental Conditions: solvent, benzonitrile [porphyrin} = 1-2 mV [TBAClO₄] = 0.1 M; scan rate 100-1000 V/s; reference electrode, Ag wire. E_{1/2} values reported are relative to SCE; the ferrocene/ferrocenium redox couple (0.43 vs. SCE, benzonitrle) was used as the internal standard.
^{b}Signifies a 2e redox step. See FIGS. 7 and 8.

Both β-β and meso-β bridged dimers 2 and 3, which also feature exclusively meso-aryl substituents, differ markedly from 1 with respect to the degree that their frontier orbitals are perturbed relative to reference (porphinato)zinc(II) complexes. The initial step of 2's first oxidative process (FIG. 6B) is shifted only modestly (15 mV) relative to reference 9, while the second anodic step is strongly stabilized by 80 mV (Table 2). In further contrast to the cyclic voltammetric data obtained for 1, the potential of 2's first reductive process is unchanged versus that determined for reference monomer 7. Meso-β bridged dimer 3 (FIG. 7A) displays similar trends in its first oxidation process: the potential of 3's initial anodic step differs little from the one-electron (1e) oxidation potentials determined for 8 and 9, while its second anodic step is stabilized on the order of 100 mV. Notably, in contrast to β-β bridged 2, 3's first reductive process is destabilized relative to its monomeric (porphinato)zinc(II) benchmarks to a degree similar (˜−275 mV) to that evinced for meso-meso bridged 1 (Table 2).

The relative differences between the respective frontier orbital energies of 1, 2, and 3 can be explained qualitatively using a simple through-space interaction model that considers the relevant interactions of the classic, macrocycle-localized Gouterman four orbitals. See Fletcher, J. T.; Therien, M. J. J. Am. Chem. Soc. 2002, 124,4298-4311. Each of the (porphinato)zinc(II) units of face-to-face porphyrin compounds 1, 2, and 3 possess HOMOs with a₂ᵤ symmetry; this orbital has significant electron density at its meso and N pyrrolyl positions. Qualitative evaluation of the extent of out-of-phase orbital interactions between macrocycle a₂ᵤ HOMOs in 1, 2, and 3 as a function of bridging connectivity provides a rationale for the observed relative energies of the frontier orbitals of these complexes.

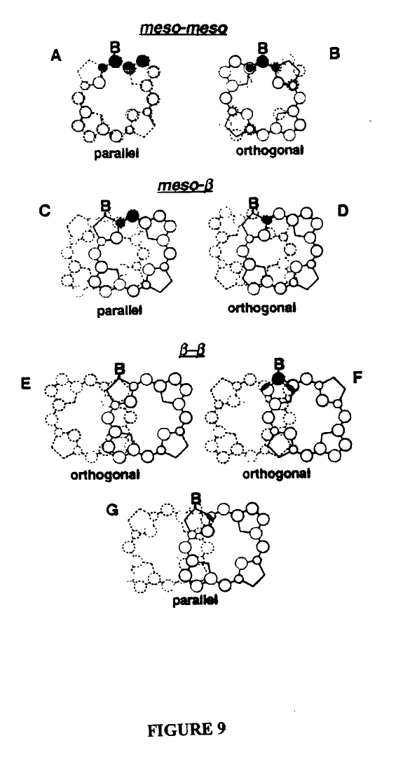

FIG. 8 identifies the nature of the highest lying filled-filled interaction as a function of macrocycle-macrocycle connectivity and the symmetries of the HOMOs of the constituent (porphinato)zinc(II) units of the cofacial porphyrin complex. The extent of wave function overlap of the HOMOs of the two (porphinato)zinc(II) units in the regions of conformational space that feature sub-van der Waals contacts between porphyrin macrocycles (assuming a 60 dihedral angle between the porphyrin least-squares plane) are highlighted in FIG. 8. As depicted in FIG. 8A, the meso-meso linked, 5,6-indanyl-bridged cofacial bis[(porphinato)zinc(II)] 1 system enforces significant a₂ᵤ-a₂ᵤ out-of-phase orbital overlap; note that, at the meso carbon atoms connected to the 5-and 6-indane positions, this unfavorable electronic interaction occurs at a distance substantially less than van der Waals contact and is thus likely a primary determinant of the extensive destabilization of the compound 1 HOMO relative to that of monomeric 8. In contrast, there is little sub-van der Waals out-of-phase orbital overlap at the bridging positions of dimer 2 (FIG. 8D); as a result, this face-to-face porphyrin compound does not display significant destabilization of its HOMO level with respect to benchmark 9. Similarly, due to minimal overlap of the (porphinato)zinc(II) a₂ᵤ HOMOs connected via a meso- linkage topology (FIG. 8B), 3 also evinces no measurable destabilization of its HOMO energy level (FIG. 9A) relative to that determined for its (porphinato)zinc(II) building blocks. Related connectivity-dependent electronic interactions have been elucidated for various classes of conjugated linear porphyrin arrays.

Each of these bis[(porphinato)zinc(II)] compounds possesses strongly stabilized second steps of their first oxidative processes, a commonly observed characteristic of cofacial porphyrin systems. It is interesting to note that the degree of stabilization (meso-meso (130 mV)>meso −(95 mV)>−(70 mV)) observed for the second steps of the first oxidative process for 1, 2, and 3 coincides with the extent of (porphinato)zinc-(porphinato)zinc HOMO-HOMO wave function overlap for a coplanar bis[(porphinato)zinc(II)] structure. This analysis is consistent with the postulate that stabilization of the second anodic step of the first oxidative process for cofacial porphyrin compounds derives from substantial electronic delocalization made possible in electrochemically generated (PZn)²⁺ species.

Disparate frontier orbital energies of the constituent (porphinato)zinc(II) units play a key role in determining the observed anodic cyclic voltammetric responses for electronically asymmetric bis[(porphinato)zinc(II)] complexes 4 and 5 (Table 2, FIG. 7). The initial anodic steps of the first oxidative processes for the respective meso-β and β-β connected structures 4 and 5 are unperturbed relative to their electron-rich monomeric components, despite the fact that for meso-β bridged 4 there exists out-of-phase overlap of the HOMOs of the constituent (porphinato)zinc(II) moieties in regions of space that feature sub-van der Waals porphyrin-porphyrin contacts (FIG. 8C). This effect has its genesis in the fact that the HOMO energy levels of the (porphinato)zinc(II) units in these cofacial structures differ by more than 600 mV (FIG. 7B). Notably, electronic delocalization effects are clearly apparent in the third oxidation for 4 and 5, with the anodic process associated primarily with the tetrakis(perfluoroalkyl)porphyrin component stabilized ˜100 mV (E_{1/2} (PZn)₂^{2+/3+}=1420 mV (4); E_{1/2} (PZn)₂^{2+/3+}=1415 mV (5)) relative to the initial oxidation of benchmark 11 (E_{1/2} (PZn)^{2+/3+}=1320 mV; see Table 2 and FIG. 7B).

It is interesting to compare the redox profile of β-β connected dimer 7 (FIG. 6C), composed of (porphinato)zinc(II) units having a₁ᵤ HOMOs with dimer 2 (FIG. 6B), which possesses (porphinato)zinc(II) components with a₂ᵤ HOMOs and an equivalent macrocycle-macrocycle linkage topology. Unlike 2, 7 shows strong destabilization of its HOMO level with respect to its monomeric benchmark complex 11. In the region of sub-van der Waals macrocycle-macrocycle contact (FIG. 8D) electron-rich β-β linked cofacial porphyrin dimer 2 shows minimal overlap of the building block (porphinato)zinc(II) HOMO wave functions; in contrast, electron-poor β-β linked dimer 7 shows significant out-of-phase (porphinato)zinc(porphinato)zinc a₁ᵤ-a₁ᵤ overlap in this region of space (FIG. 8F), giving rise to substantial electronic interactions that result in destabilization of the HOMO of the cofacial bis[(porphinato)zinc(II)] complex.

The relative energy levels of the lowest unoccupied molecular orbitals of these cofacial bis[(porphinato)metal] systems can be rationalized in a manner analogous to that used to explain differences in the observed anodic electrochemistry of these species (FIG. 9). Note that, in contrast to the case for the filled states, the extent of LUMO destabilization in the bis[(porphinato)zinc(II)] complex will correlate with the extent of in-phase macrocycle-macrocycle LUMO-LUMO interactions; such orbital overlap effects further augmentation of the already large porphyrin-porphyrin repulsive interactions, causing uniform destabilization of all occupied orbital energy levels.

The strong destabilization of the LUMO energy observed for meso-meso bridged 1 is consistent with that delineated for other cofacial porphyrin structures having meso-meso connectivity. While cathodic potentiometric data for stacked aromatic structures are sparse, it is important to note that the few literature examples of π-cofacial arene one-electron reduction potentials follow the trend delineated for meso-meso bridged cofacial bis(porphyrins). Particularly relevant in this regard is the body of data that shows that flavin moieties involved in π-stacking interactions with other aromatics display flavin-^{-/0} potentials that decrease precipitously with increasing electron-releasing character of the π-stacked arene; this behavior has been observed both in flavoenzymes and in simple model compounds.

Note that, for meso-meso bridged cofacial porphyrin complexes, significant in-phase (porphinato)metal-(porphinato)metal wave function overlap exists in the region of sub-van der Waals contact, regardless of whether the primary electronic interaction in the LUMO of the face-to-face structure involves parallel (FIG. 9A) or orthogonal (FIG. 9B) components of the e_{g} sets of the (porphinato)zinc(II) units. Similarly, meso-β bridged structures also display significant in-phase overlap of the constituent (porphinato)zinc(II)-based LUMO wave functions in the region of van der Waals contact (FIG. 7C,D). As such, meso- bridged 3, which is composed of electronically similar meso-aryl (porphinato)zinc(II) units, displays a similarly large LUMO destabilization as was observed for meso-meso bridged 1. It is interesting to note that the insightful Hunter-Sanders electrostatic model, which captures the essence of most essential trends with respect to the electronic structure of π-stacked aromatics, predicts that repulsive electrostatic interactions should be considerably less for the meso-β linkage topology with respect to that manifest by a meso-meso bridge at equivalent interplanar separations; the fact that this trend is not manifest in the potentiometric data of 1 and 3 further underscores that the relative phase relationships of the frontier orbitals of the component aromatic units is an additional factor that need also be considered in such theoretical analyses.

In contrast, electronically symmetric β-β bridged 2 and 7 each display no destabilization of their LUMO energy levels (FIG. 3), suggesting that insignificant overlap of the (porphinato)zinc(II) monomer-based e_{g} symmetric LUMO wave functions is manifest in the region of sub-van der Waals contact. Examination of the possible modes of interaction between the orthogonal and parallel e_{g} wave function components shows that two combinations (FIG. 9F and G) result in partial wave function overlap in the region of sub-van der Waals contact, while one combination of (porphinato)zinc(II)-localized orthogonal e_{g} wave functions (FIG. 9E) results in a cofacial bis[(porphinato)zinc(II)] LUMO having no electronic interaction between the porphyrin units. Potentiometric data are thus consistent with the orbital interaction shown in FIG. 9E being relevant to the description of the radical anion states of 2 and 7.

Notably, the LUMOs for both cofacial bis[(porphinato)zinc(II)] complexes 4 and 5, which feature significant macrocycle-macrocycle electronic asymmetry, show similar degrees of destabilization with respect to the potentiometrically determined LUMO energy level of the [5,10,15,20-tetrakis(perfluoroalkyl)porphinato]zinc(II) benchmark 11. The cathodic potentiometric data obtained for meso-β linked 4 (FIG. 7, Table 2) follow the trend expected for this macrocycle-macrocycle linkage topology (FIG. 9C,D) with the measured E_{1/2} (PZn)^{-/0} value destabilized 290 mV relative to the E_{1/2} (PZn)^{-/0} potential (FIG. 9, Table 2). Interestingly, β-β linked 5 displays an E_{1/2} (PZn)^{-/0} value (Table 2) destabilized by 200 mV relative to 11; this potentiometric behavior contrasts that elucidated for β-β bridged 2 and 7 (vide supra), in which the E_{1/2} (PZn)^{-/0} values are unperturbed relative to their respective standards 9 and 11 (FIG. 8, Table 2). While the origin of this behavior is an open question, it likely derives from the strong electronic asymmetry inherent to 4 and 5. As noted above, cyclic voltammetric data argue that the orbital interactions displayed in FIG. 9E describe the essential characteristics of the LUMO for electronically symmetric cofacial bis[(porphinato)zinc(II)] compounds 2 and 7. Because the HOMO and LUMO energy levels of [5,10,15,20-tetrakis(perfluoroalkyl)porphinato]zinc(II) complexes are lowered uniformly by ˜0.7 eV with respect to the analogous orbitals of (5,10,15,20-tetraphenylporphinato)zinc(II) species, significant charge resonance interactions in 5 may enforce a more coplanar ground-state structure than is manifest by 2 and 7. While X-ray structural data to support this hypothesis are lacking, evidence bolstering this assertion can be gleaned from the cyclic voltammetric data obtained for these species: 2 and 7 show peak-to-peak potentiometric separations (Ep values) for the initial cathodic steps of their respective first reductive processes of 145 mV, while the analogous redox process for 5 displays a Ep value of 230 mV, signaling a larger structural reorganization upon forming the radical anion state than accompanies the analogous reaction in the former species. Likewise, as charge resonance will also drive enhanced configuration interaction in 5 relative to 2 and 7, the (porphinato)zinc(II)-localized LUMO-LUMO interactions displayed in FIG. 9E-G may all contribute to description of 5's radical anion state and, thus, play a supplementary role in effecting net destabilization of 5's E_{1/2} PZn^{-/0} value relative to the E_{1/2} PZn^{4-/0} potential determined for 11.

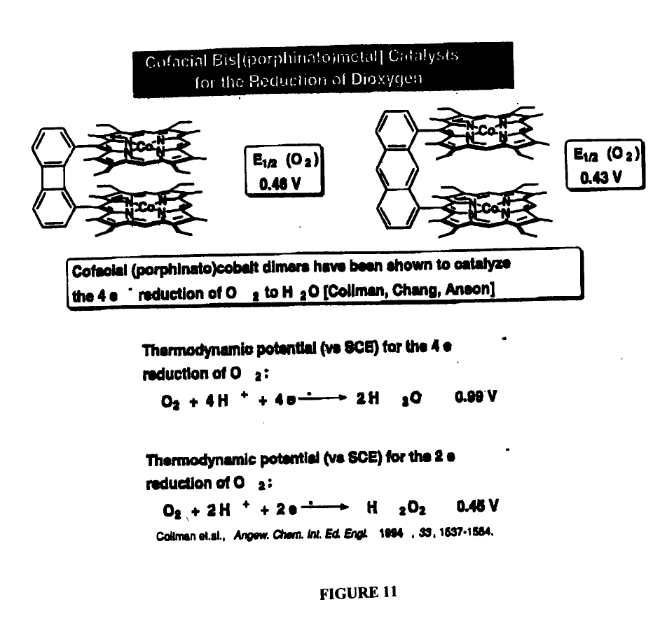

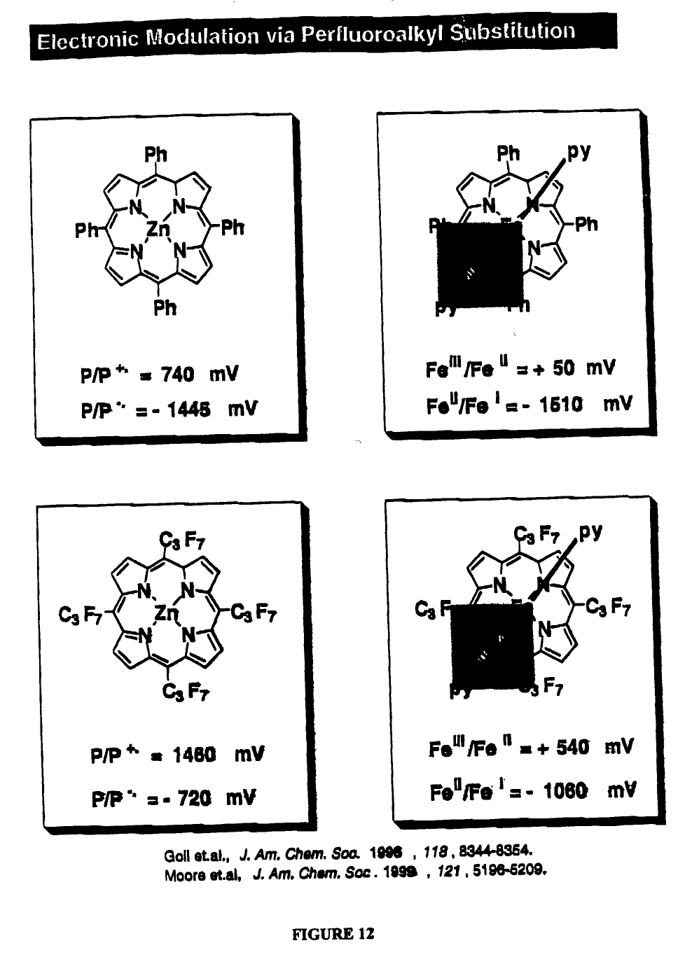

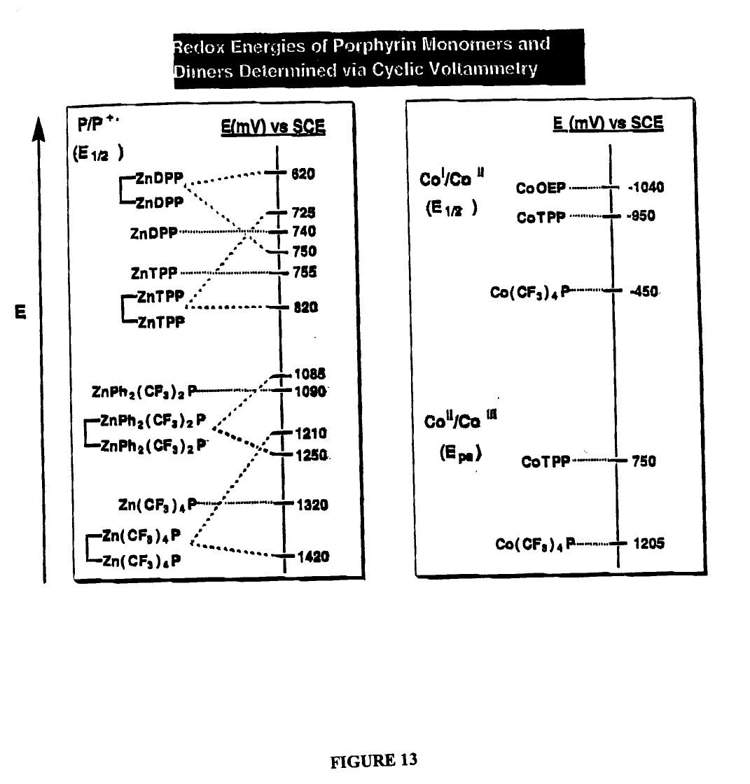

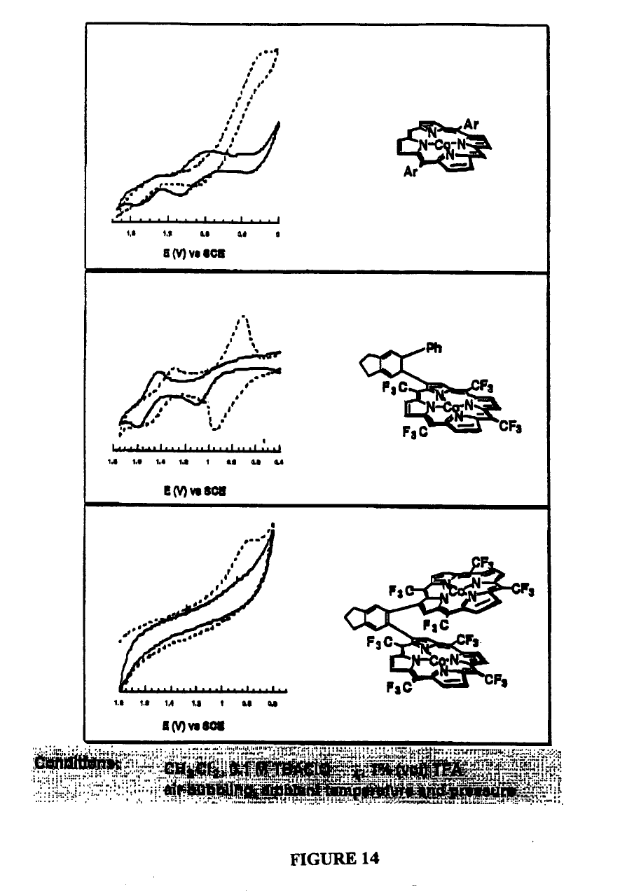

Additional electrochemical data is presented in FIGS. 11-14. FIG. 11 shows cofacial bis[(porphinato)metal] catalysts for the reduction of dioxygen. FIG. 12 shows electric modulation via perfluoroalkyl substitution. FIG. 13 is a schematic of redox energies of porphyrin monomers and dimers. These redox energies were determined by cyclic voltammetry. Finally, FIG. 14 demonstrates the changes observed in cyclic voltammetric responses upon dioxygen binding.

### EXAMPLE 32

Bis[(5,5′-10,20-Di[4-(3-Methoxy-3-Methylbutoxy)Phenyl]Porphinato)Zinc(II)]Ethyne (12).

(5-Bromo-10,20-di[4-(3-methoxy-3-methylbutoxy)phenyl]porphinato)zinc(II) (53 mg, 0.063 mmol), (5-ethynyl-10,20-di[4-(3-methoxy-3-methylbutoxy)phenyl]porphinato)zinc(II) (45 mg, 0.058 mmol), Pd₂(dba)₃ (9 mg, 0.0093 mmol), and AsPh₃ (23 mg, 0.074 mmol) were added to a 50 mL Schlenk tube under N₂. THF (10 mL) and triethylamine (2 mL) were added via syringe, giving a deep green solution which was stirred for 3 h at 35 C. The resulting green-brown solution was evaporated and the residue purified via chromatography (silica gel, 1/1 hexanes/THF). The green-brown band was collected and the solvent evaporated. The recovered solid was purified further via size-exclusion chromatography (SX-1 biobeads, THF). The dark green band was isolated, giving desired product 12. Isolated yield=83 mg (93% based on 45 mg of the porphyrin starting material).

1H NMR (250 MHz, 50/1 CDCl₃/d₅-pyridine): 10.45 (d, J=4.53 Hz, 4H,), 10.04 (s, 2H, meso), 9.24 (d, J=4.43 Hz, 4H,), 9.15 (d, J=4.43 Hz, 4H,), 8.98 (d, J=4.35 Hz, 4H,), 8.16 (d, J=8.43 Hz, 8H, Ph), 7.31 (d, J=8.53 Hz, 8H, Ph), 4.38 (t, J=7.26 Hz, 8H, CH₂), 3.33 (s, 12H, OCH₃), 2.22 (t, J=6.98 Hz, 8H, CH₂), 1.37 (s, 24H, CH₃). Vis (THF): 403 (5.08), 411 (5.08), 430 (5.00), 478 (5.46), 548 (4.21), 565 (4.18), 701 (4.69). MS (MALDI-TOF) m/z: 1535 (calcd for C₉₀H₈₆N₈O₈Zn₂ 1535).

### EXAMPLE 33

5,6-Bis[(5,5′-10,20-Di[4-(3-Methoxy-3-Methylbutoxy)Phenyl]Porphinato)Zinc(II)]Indane (25).

A 50 mL Schlenk tube was charged with 12 (50 mg, 33 mol), Co₂(CO)₈ (11 mg, 33 mol), dioxane (2.5 mL), and toluene (10 mL) under N₂. The resulting green solution was heated to 100 C, following which 10 mL of a toluene solution containing 1,6-heptadiyne (38 L, 330 mol) and Co₂(CO)₈ (11 mg, 33 mol) were added dropwise over an 18 h period. After the addition was complete, the solution was evaporated and the residue purified by chromatography (3/2 hexanes/THF). The red band was isolated, giving desired product 25. Isolated yield=50 mg (93% based on 50 mg of the porphyrin starting material).

1H NMR (250 MHz, d₅-pyridine): 10.31 (d, J=4.65 Hz, 4H,), 10.11 (s, 2H, meso), 9.29 (d, J=4.50 Hz, 4H,), 9.13 (d, J=4.68 Hz, 4H,), 8.95 (d, J=4.48 Hz, 4H,), 8.64 (s, 2H, In CH), 8.02 (d, J=8.28 Hz, 4H, Ph), 7.66 (d, J=8.25 Hz, 4H, Ph), 7.32 (d, J=8.33 Hz, 4H, Ph), 7.25 (d, J=8.45 Hz, 4H, Ph), 4.43 (t, J=7.06 Hz, 8H, R CH₂), 3.41 (t, J=6.88 Hz, 4H, In CH₂), 3.30 (s, 12H, OCH₃), 2.40 (m, 2H, In CH₂), 2.26 (t, J=6.98 Hz, 8H, R CH₂), 1.36 (s, 24H, CH₃). Vis (THF): 411 (5.54), 434 (4.74), 558 (4.32), 589 (3.72), 599 (3.71). MS (MALDI-TOF) m/z: 1627 (calcd for C₉₇H₉₄N₈O₈Zn₂ 1627).

### EXAMPLE 34

(5-Ethynyl-15-Triisopropylsilylethynyl-10,20-Di[4-(3 -Methoxy-3-Methylbutoxy)Phenyl]Porphinato)Zinc(II) (26).

(5,15-Dibromo-10,20-di[4-(3-methoxy-3 -methylbutoxy)phenyl]porphinato)zinc(II) (300 mg, 0.327 mmol), triisopropylsilylacetylene (0.47 mL, 2.094 mmol), trimethylsilylacetylene (0.10 mL, 0.698 mmol), Pd(PPh₃)₂Cl₂ (24 mg, 0.035 mmol), and CuI (7 mg, 0.035 mmol) were added to a 50 mL Schlenk tube under N₂. THF (10 mL) and triethylamine (3 mL) were transferred to the Schlenk tube via syringe; the resulting deep green solution was stirred for 24 h at 45 C. Over the course of the reaction, the solution became increasingly fluorescent when exposed to long wavelength UV irradiation from a hand-held lamp. After evaporation of volatiles, the residue was purified via chromatography (silica gel, 3/1 hexanes/THF). The dark green band was collected, which contained a mixture of three porphyrinic products. This mixture was dissolved in 50 mL of 1/1 THF/methanol, following which 1 M aqueous NaOH (2 mL) was added. After stirring 5 min at room temperature, the reaction was partitioned between CH₂Cl₂ and water, and the organic layer evaporated. The resulting mixture was purified via chromatography (silica gel, 7/3 hexanes/THF). The second dark green band isolated corresponded to the desired product 26. Isolated yield=138 mg (44% based on 300 mg of the porphyrin starting material).

1H NMR (250 MHz, 50/1 CDCl₃/d₅-pyridine): 9.65 (d, J=4.55 Hz, 2H,), 9.60 (d, J=4.57 Hz, 2H,), 8.86 (d, J=4.56 Hz, 2H,), 8.85 (d, J=4.56 Hz, 2H,), 8.02 (d, J=8.57 Hz, 4H, Ph), 7.24 (d, J=8.58 Hz, 4H, Ph), 4.34 (t, J=7.16 Hz, 4H, CH₂), 4.10 (s, 1H, CCH), 3.32 (s, 6H, OCH₃), 2.20 (t, J=7.13 Hz, 4H, CH₂), 1.39 (s, 21H, TIPS), 1.35 (s, 12H, CH₃). Vis (THF): 434 (5.50), 446 (5.35), 538 (3.48), 582 (4.06), 632 (4.46). HRMS (ESI+) m/z: 960.396104 (calcd for C57H64N4O4SiZn 960.398831). The first dark green band isolated corresponded to the (5,15-bis[triisoproplysilylethynyl]-10,20-di[4-(3-methoxy-3-methylbutoxy)phenyl]porphinato)zinc(II) side product. Isolated yield=49 mg (13% based on 300 mg of the porphyrin starting material).

1H NMR (250 MHz, 50/1 CDCl₃/d₅-pyridine): 9.63 (d, J=4.50 Hz, 4H,), 8.84 (d, J=4.44 Hz, 4H,), 8.01 (d, J=8.59 Hz, 4H, Ph), 7.22 (d, J=8.59 Hz, 4H, Ph), 4.34 (t, J=7.27 Hz, 4H, CH₂), 3.32 (s, 6H, OCH₃), 2.20 (t, J=6.97 Hz, 4H, CH₂), 1.39 (s, 42H, TIPS), 1.35 (s, 12H, CH₃). The third dark green band isolated corresponded to the (5,15-diethynyl-10,20-di[4-(3-methoxy-3-methylbutoxy)phenyl]porphinato)zinc(I) side product. Isolated yield=110 mg (42% based on 300 mg of the porphyrin starting material).

1H NMR (250 MHz, 50/1 CDCl₃/d₅-pyridine): 9.62 (d, J=4.62 Hz, 4H,), 8.86 (d, J=4.65 Hz, 4H,), 8.02 (d, J=8.60 Hz, 4H, Ph), 7.24 (d, J=8.59 Hz, 4H, Ph), 4.34 (t, J=7.15 Hz, 4H, CH₂),4.11 (s, 2H, CCH), 3.31 (s, 6H, OCH₃), 2.20 (t, J=7.15 Hz, 4H, CH₂), 1.35 (s, 12H, CH₃).

### EXAMPLE 35

(5-Triisoproplysilylethynyl-15,15′-Bis[(10,20-Di[4-(3-Methoxy-3-Methylbutoxy)Phenyl]Porphinato)Zinc(II)])Ethyne (27).

(5-Bromo-10,20-di[4-(3-methoxy-3-methylbutoxy)phenyl]porphinato)zinc(II) (107 mg, 0.128 mmol), 5 (113 mg, 0.118 mmol), Pd₂(dba)₃ (17 mg, 0.019 mmol), and AsPh₃ (46 mg, 0.15 mmol) were added to a 100 mL Schlenk tube under N₂. THF (15 mL) and triethylamine (3 mL) were added via syringe, giving a green solution which was stirred for 4 h at 40 C. Following evaporation of volatiles, the residue was purified via chromatography (silica gel, 1/1 hexanes/THF). A dark green-brown band was collected, which was purified further via size-exclusion chromatography (SX-1 biobeads, THF), giving desired product 27. Isolated yield=0.186 g (92% based on 113 mg of starting material 26).

1H NMR (250 MHz, 50/1 CDCl₃/d₅-pyridine): 10.40 (d, J=4.60 Hz, 2H,), 10.34 (d, J=4.55 Hz, 2H,), 10.03 (s, 1H, meso), 9.65 (d, J=4.58 Hz, 2H,), 9.22 (d, J=4.50 Hz, 2H,), 9.13 (d, J=4.58 Hz, 2H,), 9.02 (d, J=4.53 Hz, 2H,), 8.96 (d, J=4.45 Hz, 2H,), 8.88 (d, J=4.53 Hz, 2H,), 8.14 (d, J=8.63 Hz, 4H, Ph), 8.09 (d, J=8.70 Hz, 4H, Ph), 7.29 (d, J=8.50 Hz, 4H, Ph), 7.26 (d, J=8.58 Hz, 4H, Ph), 4.37 (t, J=7.12 Hz, 4H, CH₂), 4.36 (t, J=7.12 Hz, 4H, CH₂), 3.31 (s, 12H, OCH₃), 2.20 (t, J=7.12 Hz, 8H, CH₂), 1.40 (m, 21H, TIPS), 1.35 (s, 24H, CH₃). Vis (THF): 412 (5.13), 423 (5.13), 448 (5.01), 482 (5.48), 558 (4.26), 727 (4.84). MS (MALDI-TOF) m/z: 1715 (calcd for C₁₀₁H₁₀₆N₈O₈SiZn₂ 1714).

### EXAMPLE 36

5-[(5′-15′-Triisopropylsilylethynyl-10′,20′-Di[4-(3-Methoxy-3-Methylbutoxy)Phenyl]Porphinato)Zinc(II)]-6-[(5″-10″,20″-Di[4-(3-Methoxy-3-Methylbutoxy)Phenyl]Porphinato)Zinc(II)]Indane (28).

A 50 mL Schlenk tube was charged with 27 (80 mg, 47 mol), Co₂(CO)₈ (25 mg, 71 mol), dioxane (2.5 mL), and toluene (10 mL). The resulting green solution was heated to 100 C, following which 5 mL of a toluene solution containing 1,6-heptadiyne (54 L, 470 mol) were added dropwise over a 17 h period. After the addition was complete, the solution was evaporated and the residue purified by chromatography (silica gel, 7/3 hexanes/THF). The purple-green band was isolated giving the desired product 28. Isolated yield=58 mg (68% based on 80 mg of the porphyrin starting material).

1H NMR (250 MHz, 50/1 CDCl₃/d₅-pyridine): 9.73 (s, 1H, meso), 9.65 (d, J=4.67 Hz, 2H,), 9.60 (d, J=4.69 Hz, 2H,), 9.38 (d, J=4.43 Hz, 2H,), 8.97 (d, J=4.57 Hz, 2H,), 8.66 (d, J=4.42 Hz, 2H,), 8.63 (d, J=4.40 Hz, 2H,), 8.55 (d, J=4.51 Hz, 2H,), 8.49 (d, J=4.68 Hz, 2H,), 8.33 (s, 1H, In CH), 8.30 (s, 1H, In CH), 7.75 (m, 4H, Ph), 7.50 (m, 4H, Ph), 7.11 (m, 8H, Ph), 4.31 (t, J=7.04 Hz, 4H, R CH₂),4.30 (t, J=6.88 Hz, 4H, R CH₂), 3.43 (t, J=6.99 Hz, 4H, In CH₂), 3.31 (s, 12H, OCH₃), 2.55 (m, 2H, In CH₂), 2.19 (t, J=7.11 Hz, 4H, R CH₂), 2.17 (t, J=7.11 Hz, 4H, R CH₂), 1.35 (s, 24H, CH₃), 1.27 (s, 21H, TIPS). Vis (THF): 416 (5.60), 443 (5.04), 557 (4.30), 572 (4.28), 623 (4.28). MS (MALDI-TOF) m/z: 1806 (calcd for C₁₀₈H₁₁₄N₈O₈SiZn₂ 1807). A trailing brown band was also isolated, which corresponded to pure starting material 27. Isolated yield=26 mg (32% recovery based on 80 mg of the porphyrin starting material).

### EXAMPLE 37

5-[(5′-15′-Ethynyl-10′,20′-Di[4-(3-Methoxy-3-Methylbutoxy)Phenyl]Porphinato)Zinc(II)]-6-[(5″- 10″,20″-Di[4-(3-Methoxy-3-Methylbutoxy)Phenyl]Porphinato)Zinc(II)]Indane (29).

A 50 mL Schlenk tube was charged with 28 (58 mg, 0.032 mmol), THF (5 mL), and tetrabutylammonium fluoride (1.0 M in THF, 0.068 mL, 0.068 mmol). After stirring 10 min at room temperature, CHCl₃ (100 mL) was added to the reaction. The resulting solution was washed with NaHCO₃ (aq), following which the organic layer was separated, evaporated, and purified via chromatography (silica gel, 3/2 hexanes/THF). The purple-brown band was isolated, giving the desired product 29. Isolated yield=44 mg (83% based on 58 mg of the porphyrin starting material).

1H NMR (250 MHz, 50/1 CDCl₃/d₅-pyridine): 9.74 (s, 1H, meso), 9.65 (d, J=4.65 Hz, 2H,), 9.62 (d, J=4.62 Hz, 2H,), 9.35 (d, J=4.68 Hz, 2H,), 8.98 (d, J=4.48 Hz, 2H,), 8.66 (d, J=4.46 Hz, 2H,), 8.62 (d, J=4.59 Hz, 2H,), 8.56 (d, J=4.64 Hz, 2H,), 8.50 (d, J=4.64 Hz, 2H,), 8.34 (s, 1H, In CH), 8.32 (s, 1H, In CH), 7.76 (m, 4H, Ph), 7.50 (m, 4H, Ph), 7.12 (m, 8H, Ph), 4.32 (t, J=7.12 Hz, 8H, R CH₂), 3.91 (s, 1H, CCH), 4.31 (t, J=7.16 Hz, 8H, R CH₂), 3.45 (t, J=7.16 Hz, 4H, In CH₂), 3.32 (s, 12H, OCH₃), 2.56 (m, 2H, In CH₂), 2.19 (t, J=7.12 Hz, 8H, R CH₂), 1.37 (s, 24H, CH₃). Vis (THF): 416 (5.60), 443 (5.04), 557 (4.30), 572 (4.28), 623 (4.28). MS (MALDI-TOF) m/z: 1651 (calcd for C₉₉H₉₄N₈O₈Zn₂ 1651).

### EXAMPLE 38

5-(5′-[15′,15″-Bis(10″,20″-Di[4-(3-Methoxy-3-Methylbutoxy)Phenyl]Porphinato)Zinc(II)]Ethyne)-6-[(5′″-10′″,20′″-Di[4-(3-Methoxy-3-Methylbutoxy)Phenyl]Porphinato)Zinc(II)]Indane (30).

(5-Bromo-10,20-di[4-(3-methoxy-3-methylbutoxy)phenyl]porphinato)zinc(II) (17 mg, 0.0203 mmol), 29 (28 mg, 0.0170 mmol), Pd₂(dba)₃ (3 mg, 0.0033 mmol), and AsPh₃ (8 mg, 0.0262 mmol) were added to a 50 mL Schlenk tube under N₂. THF (5 mL) and triethylamine (1 mL) were added via syringe, and the reaction was stirred for 16 h at 45 C. Following evaporation of volatiles, the residue was purified via chromatography (silica gel, 3/2 hexanes/THF). The brown band was isolated giving desired product 30. Isolated yield=32 mg (78% based on 28 mg of starting material 29).

1H NMR (250 MHz, 50/1 CDCl₃/d₅-pyridine): 10.27 (d, J=4.55 Hz, 2H,), 10.10 (d, J=4.50 Hz, 2H,), 9.99 (s, 1H, meso), 9.74 (s, 1H, meso), 9.70 (d, J=4.68 Hz, 2H,),9.63 (d, J=4.70 Hz, 2H,), 9.19 (d, J=4.43 Hz, 2H,), 9.03 (d, J=4.55 Hz, 2H,), 8.99 (d, J=4.40 Hz, 2H,), 8.92 (d, J=4.33 Hz, 2H,), 8.74 (d, J=4.58 Hz, 2H,), 8.68 (d, J=4.43 Hz, 2H,), 8.66 (d, J=4.65 Hz, 2H,), 8.54 (d, J=4.65 Hz, 2H,), 8.36 (s, 1H, In CH), 8.35 (s, 1H, In CH), 8.08 (d, J=8.28 Hz, 4H, Ph), 7.81 (m, 4H, Ph), 7.57 (m, 4H, Ph), 7.25 (d, J=7.40 Hz, 4H, Ph), 7.14 (m, 8H, Ph), 4.34 (t, J=7.04 Hz, 4H, R CH₂), 4.33 (t, J=7.09 Hz, 4H, R CH₂), 4.32 (t, J=7.05 Hz, 4H, R CH₂), 3.46 (m, 4H, In CH₂), 3.33 (s, 6H, OCH₃), 3.32 (s, 6H, OCH₃), 3.31 (s, 6H, OCH₃), 2.57 (m, 2H, In CH₂), 2.20 (t, J=7.03 Hz, 4H, R CH₂), 2.19 (t, J=7.00 Hz, 8H, R CH₂), 1.38 (s, 12H, CH₃), 1.37 (s, 12H, CH₃), 1.35 (s, 12H, CH₃). Vis (THF): 411 (5.44), 487 (5.27), 557 (4.41), 714 (4.70). MS (MALDI-TOF) m/z: 2406 (calcd for C₁₄₃H₁₃₈N₁₂O₁₂Zn₃ 2407).

### EXAMPLE 39

Bis[(5,5′-15-Triisopropylsilylethynyl-10,20-Di[4-(3-Methoxy-3-Methylbutoxy)Phenyl]Porphinato)Zinc(II)]Ethyne (31).

A 50 mL Schlenk tube was charged with 26 (0.152 g, 0.150 mmol), 5 (0.130 mg, 0.135 mmol), Pd₂(dba)₃ (0.020 g, 0.022 mmol), AsPh₃ (0.053 g, 0.172 mmol), THF (15 mL), and triethylamine (3 mL), giving a deep green solution, which was stirred for 4 h at 40 C. Following evaporation of the solvent, the residue was purified via chromatography (silica gel, 3/2 hexanes/THF). The green-brown band was isolated, giving desired product 31. Isolated yield=0.235 g (92% based on 0.130 g of starting material 5).

1H NMR (250 MHz, 50/1 CDCl₃/d₅-pyridine): 10.29 (d, J=4.53 Hz, 4H,), 9.65 (d, J=4.60 Hz, 4H,), 9.01 (d, J=4.55 Hz, 4H,), 8.85 (d, J=4.58 Hz, 4H,), 8.09 (d, J=8.43 Hz, 8H, Ph), 7.27 (d, J=8.63 Hz, 8H, Ph), 4.36 (t, J=7.09 Hz, 8H, CH₂), 3.32 (s, 12H, OCH₃), 2.20 (t, J=7.13 Hz, 8H, CH₂), 1.39 (m, 42H, TIPS), 1.36 (s, 24H, CH₃). Vis (THF): 424 (5.22), 436 (5.20), 488 (5.47), 576 (4.25), 747 (4.91). MS (MALDI-TOF) m/z: 1895 (calcd for C₁₁₂H₁₂₆N₈O₈Si₂Zn₂ 1895).

### EXAMPLE 40

5,6-Bis[(5′-15′-Triisopropylsilylethynyl-10′,20′-Di[4-(3-Methoxy-3-Methylbutoxy)Phenyl]Porphinato)Zinc(II)]Indane (32).

A 50 mL Schlenk tube was charged with 31 (65 mg, 34.3 mol), Co₂(CO)8 (18 mg, 51.5 mol), dioxane (2.5 mL), and toluene (10 mL). The resulting brown solution was heated to 100 C, following which 5 mL of a toluene solution containing 1,6-heptadiyne (40 L, 343 mol) were added dropwise over a 17 h period. After the addition was complete, the resulting green solution was evaporated and the residue purified by chromatography (silica gel, 7/3 hexanes/THF). The green band was isolated giving the desired product 32. Isolated yield=50 mg (73% based on 65 mg of the porphyrin starting material).

1H NMR (250 MHz, 50/1 CDCl₃/d₅-pyridine): 9.56 (d, J=4.71 Hz, 4H,), 9.40 (d, J=4.45 Hz, 4H,), 8.57 (d, J=4.44 Hz, 4H,), 8.50 (d, J=4.70 Hz, 4H,), 8.29 (s, 2H, In CH), 7.75 (m, 4H, Ph), 7.48 (m, 4H, Ph), 7.11 (m, 8H, Ph), 4.31 (t, J=7.13 Hz, 8H, R CH₂), 3.44 (t, J=7.00 Hz, 4H, In CH₂), 3.30 (s, 12H, OCH₃), 2.55 (m, 2H, In CH₂), 2.20 (t, J=7.08 Hz, 8H, R CH₂), 1.37 (s, 24H, CH₃), 1.23 (m, 42H, TIPS). Vis (THF): 423 (5.43), 428 (5.43), 454 (4.87), 577 (4.29), 613 (4.22), 625 (4.32). MS (MALDI-TOF) m/z: 1986 (calcd for C₁₁₉H₁₃₄N₈O₈Si₂Zn₂ 1987). A trailing brown band was also isolated, which corresponded to pure porphyrinic starting material. Isolated yield=17 mg (26% recovery based on 65 mg of compound 32).

### EXAMPLE 41

5,6-Bis[(5′,5″-15′-Ethynyl-10′,20′-Di[4-(3-Methoxy-3-Methylbutoxy)Phenyl]Porphinato)Zinc(II)]Indane (33).

A 50 mL Schlenk tube was charged with 32 (58 mg, 0.029 mmol), THF (5 mL), and tetrabutylammonium fluoride (1.0 M in THF, 0.09 mL, 0.09 mmol). After stirring 10 min at room temperature, CHCl₃ (100 mL) was added, and the resulting solution was washed with NaHCO₃ (aq). The organic layer was separated, evaporated, and purified via chromatography (silica gel, 7/3 hexanes/THF). The green band was isolated, giving desired product 33. Isolated yield=47 mg (98% based on 58 mg of the porphyrin starting material).

1H NMR (250 MHz, 50/1 CDCl₃/d₅-pyridine): 9.56 (d, J=4.52 Hz, 4H,), 9.36 (d, J=4.61 Hz, 4H,), 8.57 (d, J=4.63 Hz, 4H,), 8.50 (d, J=4.66 Hz, 4H,), 8.30 (s, 2H, In CH), 7.73 (m, 4H, Ph), 7.47 (m, 4H, Ph), 7.11 (m, 8H, Ph), 4.31 (t, J=7.12 Hz, 8H, R CH₂), 3.92 (s, 2H, CCH), 3.44 (t, J=7.14 Hz, 4H, In CH₂), 3.32 (s, 12H, OCH₃), 2.55 (m, 2H, In CH₂), 2.19 (t, J=7.12 Hz, 8H, R CH₂), 1.36 (s, 24H, CH₃). Vis (THF): 423 (5.43), 428 (5.43), 454 (4.87), 577 (4.29), 613 (4.22), 625 (4.32). MS (MALDI-TOF) m/z: 1671 (calcd for C₁₀₁H₉₄N₈O₈Zn₂ 1675).

### EXAMPLE 42

5,6-Bis(5′-15′,15″-Bis[(10′,20′-Di[4-(3-Methoxy-3-Methylbutoxy)Phenyl]Porphinato)Zinc(II)]Ethyne)Indane (34).

(5-Bromo- 10,20-di[4-(3-methoxy-3-methylbutoxy)phenyl]porphinato)zinc(II) (31 mg, 0.0370 mmol), 33 (30 mg, 0.0180 mmol), Pd₂(dba)₃ (5 mg, 0.0054 mmol), AsPh₃ (0.013 g, 0.043 mmol), THF (10 mL), and triethylamine (2 mL) were added to a 50 mL Schlenk tube. The resulting solution was stirred for 23 h at 40 C. Following evaporation of volatiles, the residue was purified via chromatography (silica gel, 1/1 hexanes/THF). The brown band was collected and purified further via size-exclusion chromatography (SX-1 biobeads, THF), giving desired product 34. Isolated yield=36 mg (63% based on 30 mg of starting material 33).

1H NMR (250 MHz, 50/1 CDCl₃/d₅-pyridine): 10.27 (d, J=4.55 Hz, 4H,), 10.13 (d, J=4.63 Hz, 4H,), 9.98 (s, 2H, meso), 9.63 (d, J=4.70 Hz, 4H,), 9.18 (d, J=4.45 Hz, 4H,), 9.02 (d, J=4.50 Hz, 4H,), 8.91 (d, J=4.45 Hz, 4H,), 8.78 (d, J=4.50 Hz, 4H,), 8.59 (d, J=4.58 Hz, 4H,), 8.36 (s, 2H, In CH), 8.07 (d, J=8.43 Hz, 8H, Ph), 7.86 (m, 4H, Ph), 7.63 (m, 4H, Ph), 7.20 (m, 16H, Ph), 4.35 (t, J=7.13 Hz, 8H, R CH₂), 4.32 (t, J=7.13 Hz, 8H, R CH₂), 3.48 (t, J=7.03 Hz, 4H, In CH₂), 3.35 (s, 12H, OCH₃), 3.29 (s, 12H, OCH₃), 2.58 (m, 2H, In CH₂), 2.22 (t, J=7.13 Hz, 8H, R CH₂), 2.19 (t, J=7.13 Hz, 8H, R CH₂), 1.40 (s, 24H, CH₃), 1.33 (s, 24H, CH₃). Vis (THF): 419 (5.39), 479 (5.35), 558 (4.51), 713 (4.85). MS (MALDI-TOF) m/z: 3182 (calcd for C₁₈₉H₁₇₈N₁₆O₁₆Zn₄ 3183).

It should be noted that Fletcher, J. T.; Therien, M. J. J. Am. Chem. Soc. 2000, 122, 12393-12394, Fletcher, J. T.; Therien, M. J. J. Am. Chem. Soc. 2002, 124, 4298-4311 and Fletcher, J. T.; Therien, M. J. Inorganic Chemistry 2002, 41, 331-341 are particularly useful to one skilled in the art. In addition to these papers, all patents, patent applications, and scientific literature referred to herein are hereby incorporated by reference in their entirety.

Those skilled in the art will appreciate that numerous changes and modifications may be made to the preferred embodiments of the invention and that such changes and modifications may be made without departing from the spirit of the invention. For example, it is believed that the methods of the present invention can be practiced using porphyrin-related compounds such as chlorins, phorbins, bacteriochlorins, porphyrinogens, sapphyrins, texaphrins, and pthalocyanines in place of porphyrins. It is also believed that, in addition to ethyne and butadiyne moities, the invention can be practiced using other moieties, including ethene, polyines, polyenes, and allene. It is therefore intended that the appended claims cover all such equivalent variations as fall within the true spirit and scope of the invention.

What is claimed:

1. 5,6-Bis(porphinato)zinc(II)indane;
wherein said porphinato group is substituted with at least on compound of the group consisting of C₁-C₁₂ alkyl, C₆-C₂₀ aryl, perhaloalkyl, C₂-C₁₂ alkenyl, and C≡C—R;
wherein R is H, C₁-C₁₂ alkyl, or C₁-C₂₀ aryl; and
said alkyl and aryl groups are optionally substituted with alkyl, aryl, alkoxy, or aryloxy.

2. A compound having formula (1), (2), or (3): 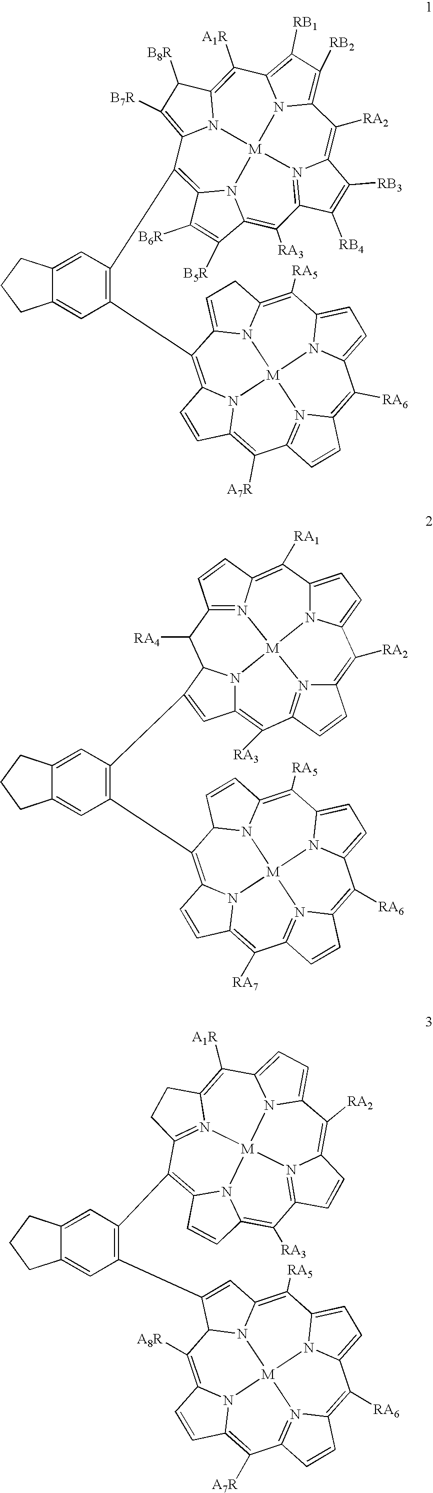
wherein M and M′ are metal ions and RB₁-RB₈ are independently selected from the group consisting of H, C₁-C₁₂ alkyl, C₆-C₂₀ aryl, perhaloalkyl, C₂-C₁₂ alkenyl, and C≡C—R;
wherein R is H, C₁-C₁₂ alkyl, or C₁-C₂₀ aryl;
wherein each alkyl or aryl group may be optionally substituted with alkyl, aryl, alkoxy, or aryloxy;
provided that at least one of the following conditions applies:
(a) at least one of RB₁-RB₈ or one of RA₁-RA₈ is perhaloalkyl; or
(b) at least one of RB₁-RB₈ or one of RA₁-RA8 is ethyne, alkynyl, oligynyl, or has the formula C≡CR, or
(c) at least one of RA₁-RA8 is electron withdrawing relative hydrogen provided at least one of RB₁-RB₈ is not H; or
(d) one of RB₁-RB₈ is electron withdrawing relative hydrogen; or
(e) one of RA₁-RA₈ is electron withdrawing relative hydrogen provided all of RA₁-RA₈ are not perfluorophenyl; or
(f) all of RA₁-RA₈ are electron withdrawing relative hydrogen provided at least one of RB₁-RB₈ is not H;
(g) at least one of RB₁-RB₈ or one of RA₁-RA₈ is halooalkyl; or
(h) at least one of RB₁-RB₈ or one of RA₁-RA₈ is ethene, ethynyl, oligoenyl, or has the formula C(R_{C})═C(R_{D})(R_{E}) where R_{C}, R_{D}, and R_{E} are independently H, C₁-C₂ alkyl, or C₁-C₂₀ aryl;

3. The compound of claim 2 wherein at least one of RB₁-RB₈ or one of RA₁-RA₈ is perhaloalkyl.

4. The compound of claim 3 wherein the perhaloalkyl is perfluroalkyl.

5. The compound of claim 2 wherein at least one of RB₁-RB₈ or one of RA₁-RA₈ is ethyne, alkynyl, oligynyl, or has the formula C≡CR.

6. The compound of claim 2, wherein said compound is capable of binding diatomic, triatomic, or tetraatomic molecules.

7. The compound of claim 2 having homogeneous or heterogeneous redox catalyst activity.

8. The compound of claim 2, wherein said compound is capable of functioning as an electrocatalyst.

9. The compound of claim 2, wherein said compound is capable of selectively oxidizing hydrogen, water, carbon monoxide, or nitric oxide.

10. The compound of claim 2, wherein said compound is capable of selectively oxidizing a hydrocarbon.

11. The compound of claim 2, wherein said compound is capable of selectively reducing dinitrogen, dioxygen, carbon monoxide, nitric oxide, or carbon dioxide.

12. An electrode wherein a compound of claim 2 is absorbed onto said electrode.

13. An electrode wherein a compound of claim 2 is covalently or noncovalently bonded to an electrode.

14. A fuel cell comprising a compound of claim 2.

15. A method comprising an oxidative or reductive transformation comprising contacting a reactant with a compound of claim 1 or 2.
